(19) United States
(12) Patent Application Publication
(10) Pub. No.: US 2020/0054833 A1
(43) Pub. Date: Feb. 20, 2020

(54) FLUID DISPENSING APPARATUS

(71) Applicant: Marcus Gregory Romanello, West Chester, OH (US)

(72) Inventor: Marcus Gregory Romanello, West Chester, OH (US)

(21) Appl. No.: 16/105,698

(22) Filed: Aug. 20, 2018

(51) Int. Cl. A61M 5/24; A61M 5/32

(52) U.S. Cl. CPC A61M 5/2466; A61M 5/3202; A61M 5/2448; A61M 2005/2433; A61M 2209/088; A61M 2005/2474; A61M 5/2422

(57) ABSTRACT

Embodiments included herein are directed towards a fluid dispensing apparatus. Embodiments may include a housing including a vial containing pre-dosed medication. The apparatus may further include an injection needle contained within a removable needle cap, the injection needle having a drug contact end and a patient contact end, the injection needle configured to puncture at both ends. The apparatus may also include a hinge operatively connecting the housing and the injection needle, wherein the hinge provides movement from a first position wherein the drug contact end is not in contact with the pre-dosed medication to a second position where the drug contact end is in contact with the pre-dosed medication. The apparatus may further include an opening associated with a proximal end of the housing configured to receive the removable needle cap.

### FIELD OF THE INVENTION

The present invention relates to a syringe and more particularly to a disposable, foldable syringe.

### DISCUSSION OF THE RELATED ART

The lifetime prevalence of anaphylaxis is 1-2% of the population as a whole and nearly 15 million Americans have food allergies. Rapid administration of drugs such as epinephrine via intramuscular injection is only treatment in life-threatening situations. However, current auto-injectors on the market are expensive and bulky due to gas and spring loaded mechanisms, excessive contained volume, and other features, limiting portability and likelihood of carry at time of anaphylactic exposure.

### SUMMARY OF DISCLOSURE

In an embodiment of the present disclosure a fluid dispensing apparatus is provided. The fluid dispensing apparatus may include a housing including a vial containing pre-dosed medication. The apparatus may further include an injection needle contained within a removable needle cap, the injection needle having a drug contact end and a patient contact end, the injection needle configured to puncture at both ends. The apparatus may also include a hinge operatively connecting the housing and the injection needle, wherein the hinge provides movement from a first position wherein the drug contact end is not in contact with the pre-dosed medication to a second position where the drug contact end is in contact with the pre-dosed medication. The apparatus may further include an opening associated with a proximal end of the housing configured to receive the removable needle cap.

One or more of the following features may be included. The apparatus may include a sterilization cap configured to cover the housing and the injection needle. The apparatus may also include a movable cover configured to restrict or allow access to an inner portion of the housing. The pre-dosed medication may be selected from the group consisting of epinephrine. The housing may be transparent. The drug contact end and the patient contact end of the needle may be beveled. At least one connector may be configured to allow a patient to wear the apparatus. The housing may be at least one of rectangular and cylindrical.

In another embodiment of the present disclosure a fluid dispensing method is provided. The method may include providing a housing including a vial containing pre-dosed medication and connecting an injection needle contained within a removable needle cap to the housing using a hinge, the injection needle having a drug contact end and a patient contact end. The method may further include moving the injection needle from a first position wherein the drug contact end is not in contact with the pre-dosed medication to a second position where the drug contact end is in contact with the pre-dosed medication. The method may also include receiving the removable needle cap at an opening associated with a proximal end of the housing.

One or more of the following features may be included. The method may include covering the housing and the injection needle with a sterilization cap. The method may further include using a movable cover configured to restrict or allow access to an inner portion of the housing. The pre-dosed medication may be selected from the group consisting of epinephrine . . . . The housing may be transparent. The drug contact end and the patient contact end of the needle may be beveled. The method may further include connecting the apparatus with a patient using at least one connector. The housing may be at least one of rectangular and cylindrical.

In yet another embodiment of the present disclosure a fluid dispensing apparatus is provided. The apparatus may include a first housing portion including an injection needle connected to a first fluid conduit and a removable needle cap and a second housing portion including a second fluid conduit. The apparatus may also include a third housing portion including a third fluid conduit, a vial containing pre-dosed medication, and a vial piercing needle tip, wherein the first housing portion is configured to rotate to connect to the second housing portion and the second housing portion is configured to rotate to connect to the third housing portion.

One or more of the following features may be included. The second housing and vial piercing tip may operate together to puncture the vial containing pre-dosed medication. The third housing may slide down the second housing to dispense fluid. Fluid may be dispensed from the third fluid conduit through the second fluid conduit and the first fluid conduit before reaching the injection needle.

### BRIEF DESCRIPTION OF THE DRAWINGS

The accompanying drawings, which are included to provide a further understanding of embodiments of the present disclosure and are incorporated in and constitute a part of this specification, illustrate embodiments of the present disclosure and together with the description serve to explain the principles of embodiments of the present disclosure.

FIG. 1 is a perspective view of a syringe according to an embodiment of the present disclosure;

FIG. 2 is a perspective view of a syringe according to an embodiment of the present disclosure;

FIG. 3 is a perspective view of a syringe according to an embodiment of the present disclosure;

FIG. 4 is a perspective view of a syringe according to an embodiment of the present disclosure;

FIG. 5 is a perspective view of a syringe and associated hinge according to an embodiment of the present disclosure;

FIG. 6 is a perspective view of a syringe in a second position according to an embodiment of the present disclosure;

FIG. 7 is a perspective view of a syringe according to an embodiment of the present disclosure;

FIG. 8 is a perspective view of a syringe according to an embodiment of the present disclosure;

FIG. 9 is a perspective view of a syringe according to an embodiment of the present disclosure;

FIG. 10 is a perspective view of a syringe according to an embodiment of the present disclosure;

FIG. 11 is a perspective view of a syringe according to an embodiment of the present disclosure;

FIG. 12 is a perspective view of a syringe according to an embodiment of the present disclosure;

FIG. 13 is a perspective view of a syringe according to an embodiment of the present disclosure;

FIG. 14 is a top view of a syringe according to an embodiment of the present disclosure;

FIG. 15 is a perspective view of a syringe according to an embodiment of the present disclosure;

FIG. 16 is a perspective view of a syringe according to another embodiment of the present disclosure;

FIG. 17 is a perspective view of a syringe according to another embodiment of the present disclosure;

FIG. 18 is a perspective view of a syringe according to another embodiment of the present disclosure;

FIG. 19 is a perspective view of a syringe according to another embodiment of the present disclosure;

FIG. 20 is a perspective view of a syringe according to an embodiment of the present disclosure;

FIG. 21 is a perspective view of a syringe according to another embodiment of the present disclosure;

FIG. 22 is a perspective view of a syringe according to another embodiment of the present disclosure;

FIG. 23 is a perspective view of a syringe according to another embodiment of the present disclosure;

FIG. 24 is a perspective view of a syringe according to another embodiment of the present disclosure;

FIG. 25 is a perspective view of a syringe according to another embodiment of the present disclosure;

FIG. 26 is a perspective view of a syringe according to another embodiment of the present disclosure;

FIG. 27 is a perspective view of a syringe according to another embodiment of the present disclosure;

FIG. 28 is a perspective view of a syringe according to another embodiment of the present disclosure;

FIG. 29 is a perspective view of a syringe according to another embodiment of the present disclosure;

FIG. 30 is a perspective view of a syringe according to an embodiment of the present disclosure;

FIG. 31 is a perspective view of a syringe according to another embodiment of the present disclosure;

FIG. 32 is a perspective view of a syringe according to another embodiment of the present disclosure;

FIG. 33 is a perspective view of a syringe according to yet another embodiment of the present disclosure;

FIG. 34 is a perspective view of a syringe according to yet another embodiment of the present disclosure;

FIG. 35 is a perspective view of a syringe according to yet another embodiment of the present disclosure;

FIG. 36 is a perspective view of a syringe according to yet another embodiment of the present disclosure;

FIG. 37 is a perspective view of a syringe according to yet another embodiment of the present disclosure;

FIG. 38 is a perspective view of a syringe according to yet another embodiment of the present disclosure;

FIG. 39 is a perspective view of a syringe according to yet another embodiment of the present disclosure;

FIG. 40 is a perspective view of a syringe according to yet an embodiment of the present disclosure;

FIG. 41 is a perspective view of a syringe according to another embodiment of the present disclosure;

FIG. 42 is a perspective view of a syringe according to yet another embodiment of the present disclosure;

FIG. 43 is a perspective view of a syringe according to yet another embodiment of the present disclosure;

FIG. 44 is a perspective view of a syringe according to yet another embodiment of the present disclosure;

FIG. 45 is a perspective view of a syringe according to yet another embodiment of the present disclosure;

FIG. 46 is a perspective view of a syringe according to yet another embodiment of the present disclosure;

FIG. 47 is a perspective view of a syringe according to yet another embodiment of the present disclosure;

FIG. 48 is a perspective view of a syringe according to yet another embodiment of the present disclosure; and

FIG. 49 is a perspective view of a syringe according to yet another embodiment of the present disclosure.

Like reference numerals may indicate like elements.

### DETAILED DESCRIPTION

As used herein, the terms “distal” or “distal end” may be used to define the part or surface of an element which is facing the patient or positioned furthest from the user as described hereinafter. The term “proximal” or “proximal end” are used herein to define the part of surface of an element which is facing away from the patient or positioned closest to the user.

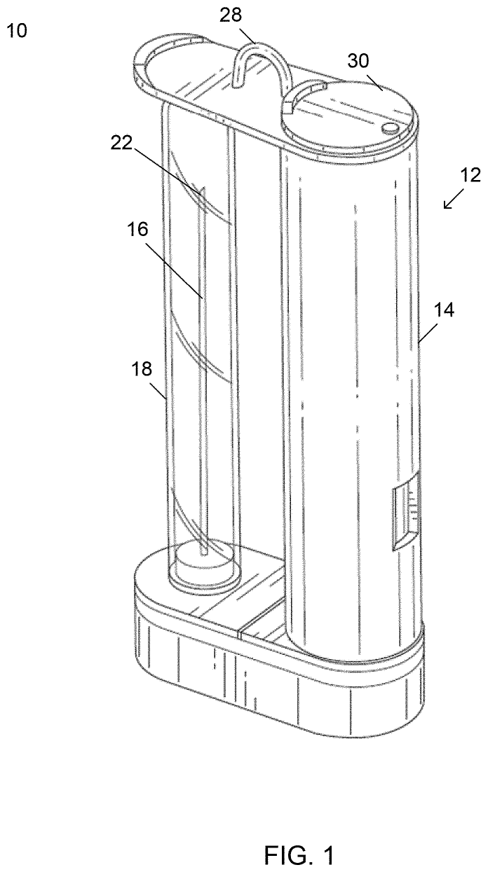

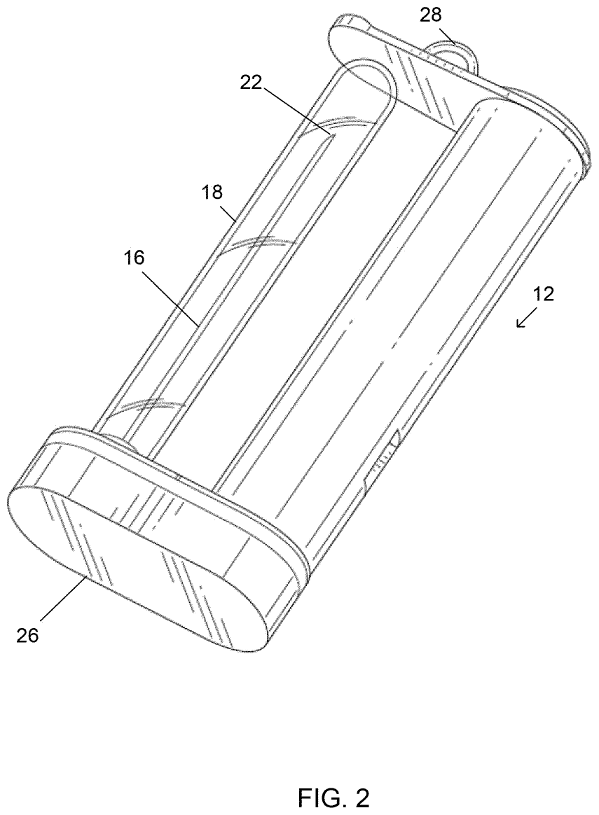

Referring now to FIGS. 1-5, the single-use syringe of the present disclosure is described herein as a fluid dispensing apparatus 10 having a housing 12, which may include a hollow elongated syringe barrel 14. Fluid dispensing apparatus 10 may also include an injection needle 16, which may be enclosed within a removable needle cap 18. Injection needle 16 may be of any suitable size, for example, 3 cm, 1-5 cm, etc.

In some embodiments, housing 12 may include a container or vial containing pre-dosed medication. The medication may be any suitable medication, including, but not limited to, epinephrine, etc. (e.g., 0.3 ml or 0.3 mg of 1:1000 epinephrine). It should be noted that this is provided merely by way of example as any other medication of any suitable dosage may be contained within the vial. The medication and its container may also account for any residual volume associated with the needle. It should be noted that housing 12 may be constructed out of any suitable material.

In some embodiments, housing 12 may be transparent and/or semi-transparent. As is discussed below in further detail, housing 12 may be of any suitable shape, including, but not limited to, rectangular, cylindrical, etc. In some embodiments, the shape of housing 12 may be configured to mate with that of removable needle cap 18, which may act as a plunger once removed.

As discussed above, injection needle 16 may be contained within removable needle cap 18. Injection needle 16 may have a drug contact end 20 and a patient contact end 22. Accordingly, injection needle 16 may be configured to puncture at both ends. Additionally and/or alternatively, both the drug contact end and the patient contact end of injection needle 16 may be beveled. Drug contact end 20 may be configured to pierce a membrane associated with a vial or container of medication. The vial may be stored within a compartment that is associated with housing 12.

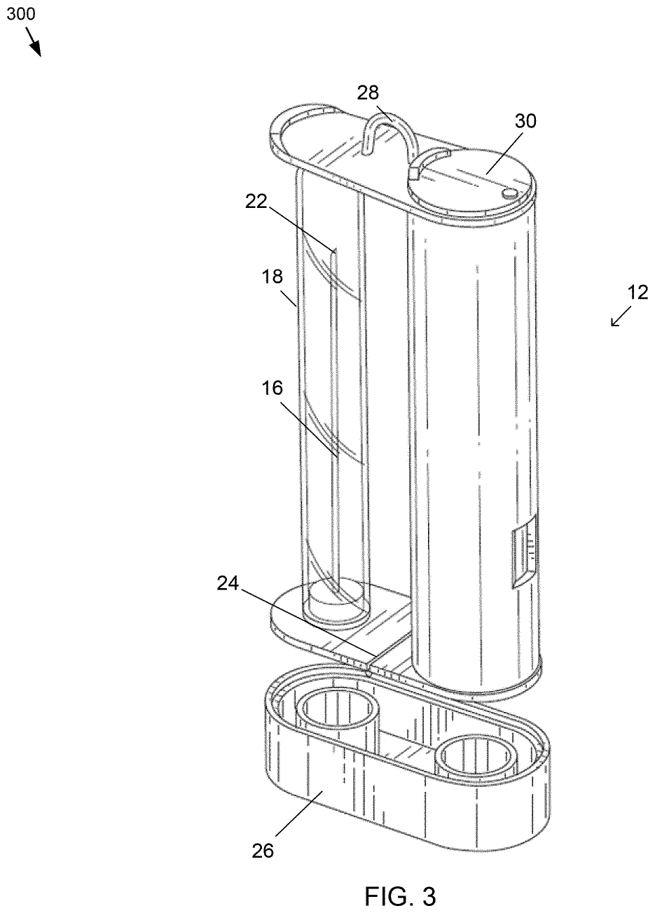

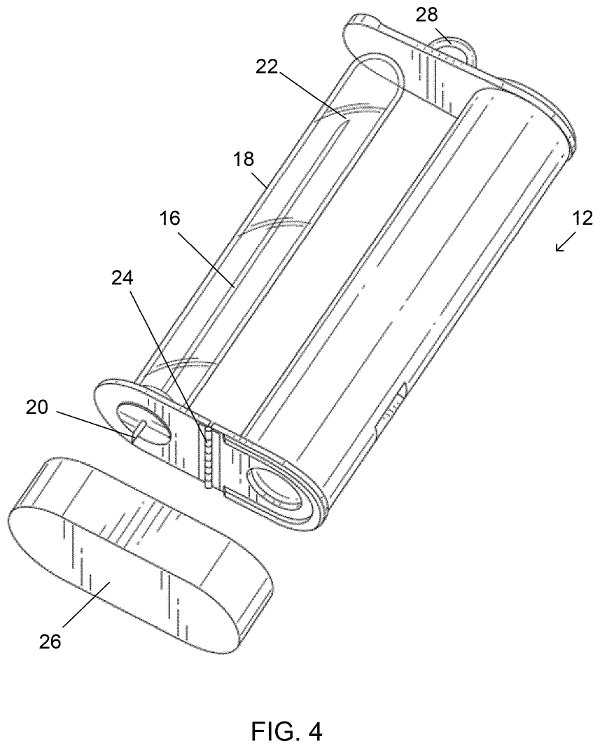

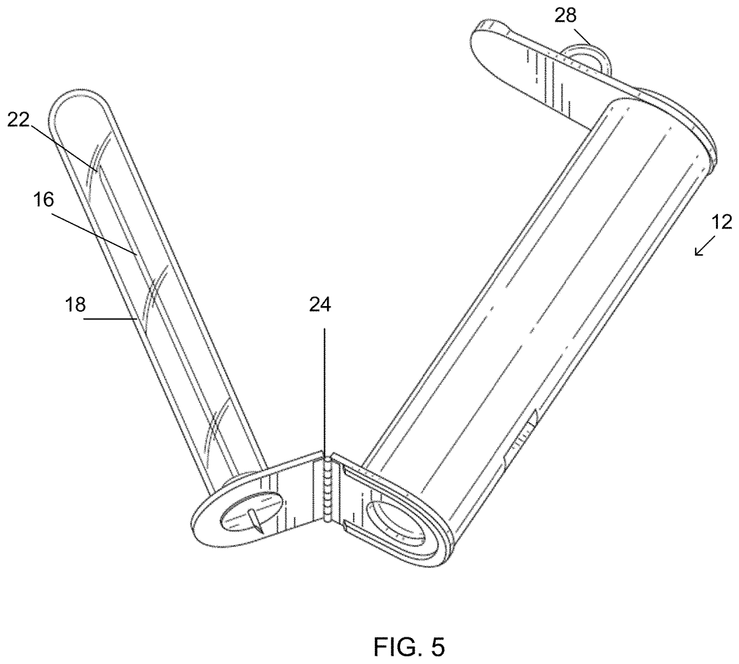

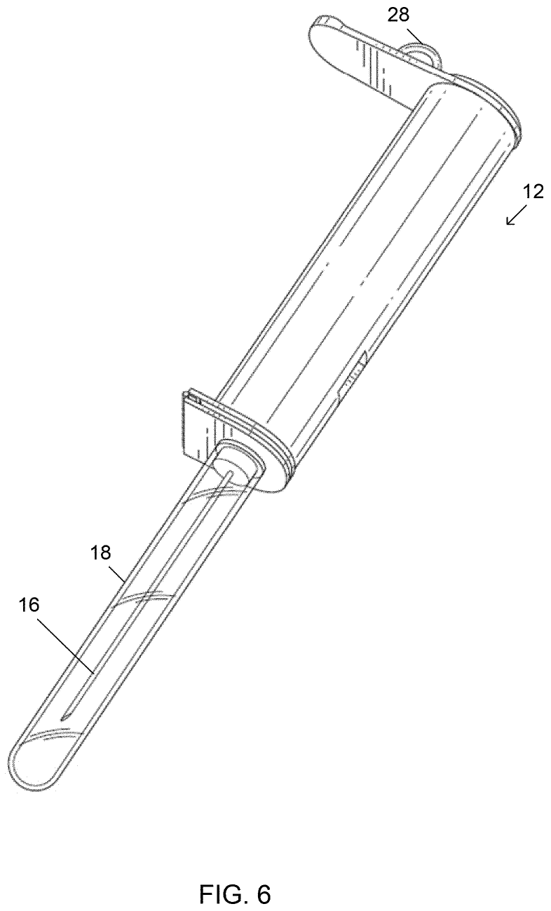

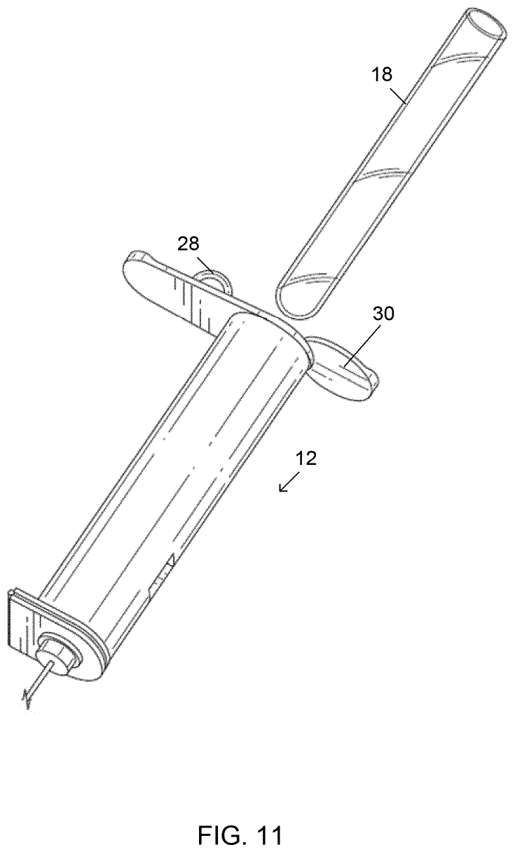

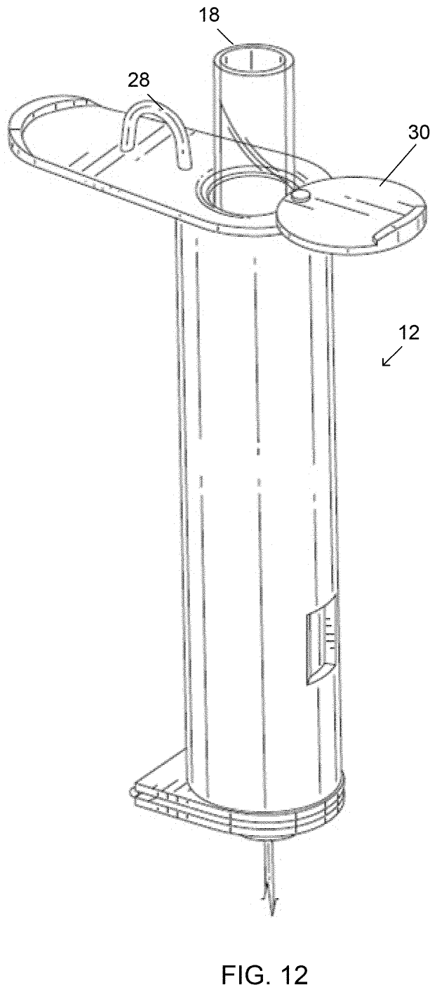

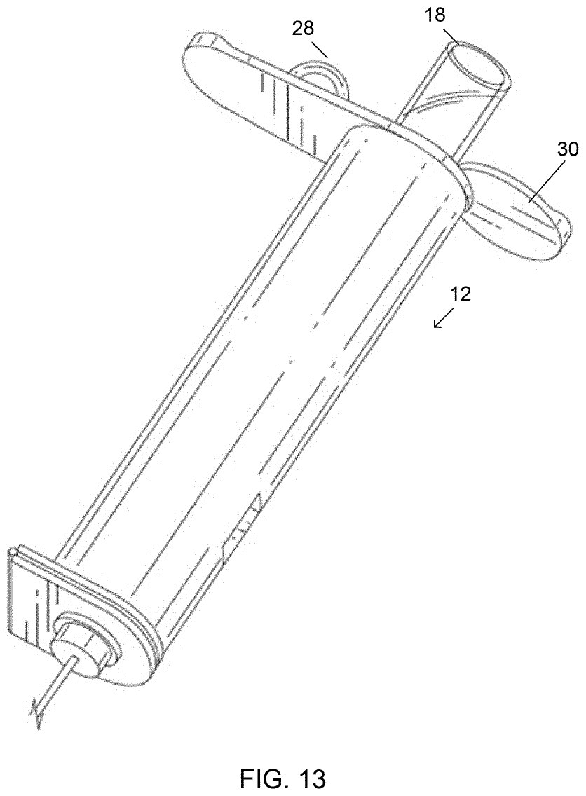

In some embodiments, and referring again to FIGS. 3-15, fluid dispensing apparatus 10 may include hinge 24 that may be configured to connect housing 12 with injection needle 16. In operation, hinge 24 may operatively connect housing 12 and injection needle 16 and hinge 24 may be configured to allow for movement from a first position (e.g., as shown in FIG. 4) wherein drug contact end 20 is not in contact with the pre-dosed medication to a second position (e.g., as shown in FIG. 6) where drug contact end 20 is in contact with the pre-dosed medication, which may be contained within a vial or other containing device. In some embodiments, hinge 24 may allow for approximately 180° of movement from the first position to the second position. FIG. 5 shows a transition from the first position to the second position.

In some embodiments, and referring again to FIGS. 3-4, a removable sterilization cap 26 may be included at a first end of fluid dispensing apparatus 10 and at least one connector 28 may be located at a second end of fluid dispensing apparatus. Connector 28 may be configured to allow a patient to wear fluid dispensing apparatus 10. For example, the user may affix connector 28 to a necklace, wristband, etc. Other embodiments, such as keychain and/or keyring connections and the like are also within the scope of the present disclosure. Fluid dispensing apparatus 10 may also include one or more magnetic connectors to allow for various forms of attachment (e.g., wristwear, etc.). Sterilization cap 26 may be configured to cover some or all of housing 12 and/or injection needle 16. Accordingly, sterilization cap 26 may be configured to ensure the sterility of the vial membrane as well as the sterility of the piercing tip of injection needle 16.

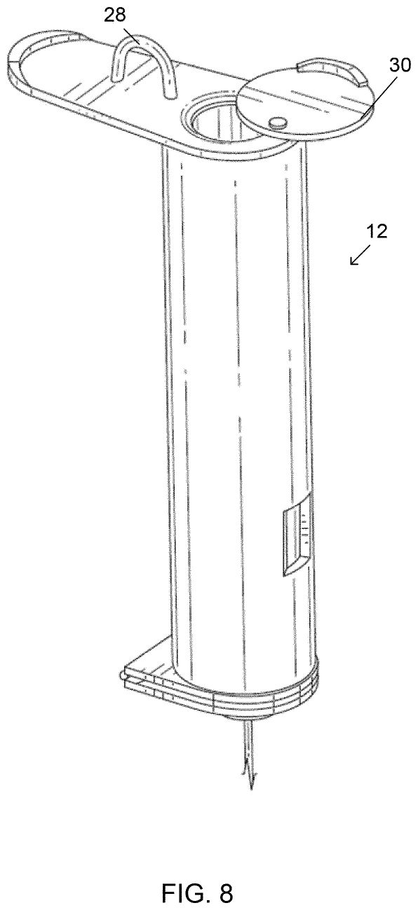

In some embodiments, fluid dispensing apparatus 10 may also include a movable cover 30 configured to restrict or allow access to an inner portion of housing 12. In operation, movable cover 30 may be swung open to generate an opening associated with a proximal end of housing 12. The opening is shown in FIGS. 8-13 and may be configured to receive removable needle cap 18, which may act as a plunger.

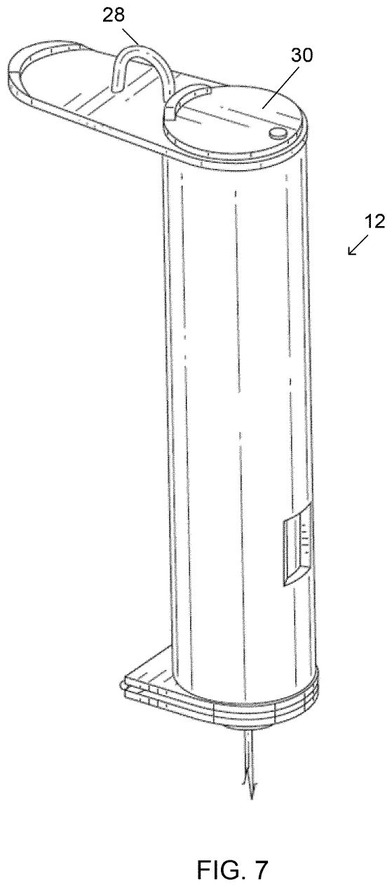

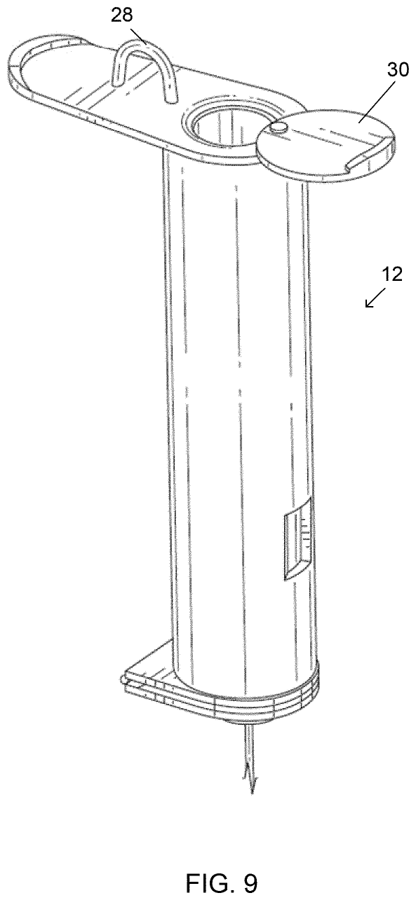

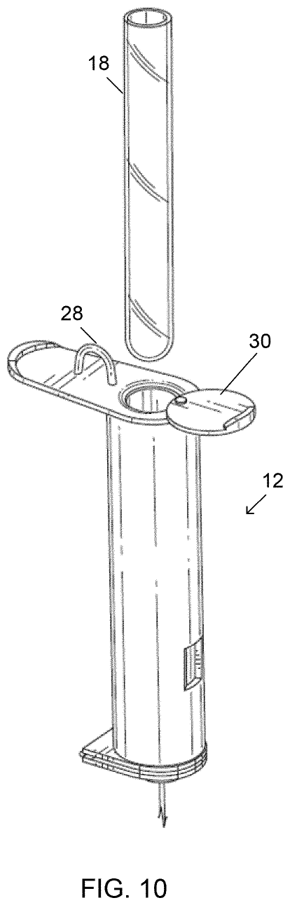

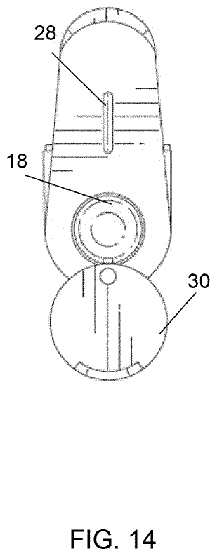

Referring again to FIG. 6, an embodiment showing fluid dispensing apparatus 10 with removable needle cap 18 and needle 16 in the second position is provided. In operation, a user may remove removable needle cap 18 from housing 12 as is shown in FIG. 7. The user may then open movable cover 30 the access the opening within housing 12 as is shown in FIGS. 8-9. Removable needle cap 18 may then be inserted into the opening of housing 12 as shown in FIGS. 10-14. Housing 12 may be configured to receive removable needle cap 18 and the interior portion of housing 12 may have a shape that corresponds to that of removable needle cap 18. FIG. 14 shows a top view of fluid dispensing apparatus 10 with the removable needle cap 18 inserted into housing 12.

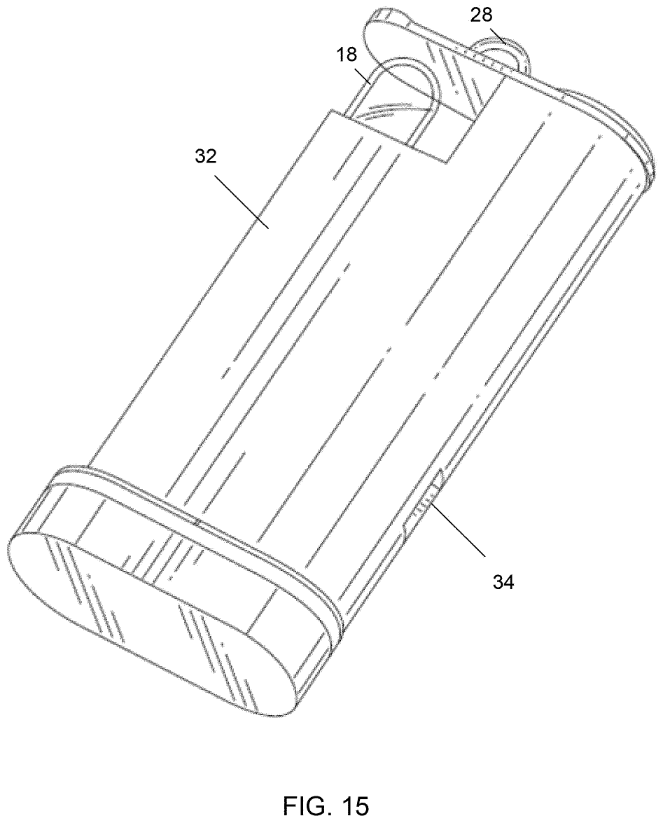

In some embodiments, and referring to FIG. 15, single use syringe may include an enclosure assembly 32, which may be configured to protect the syringe and allow for easy transport. Enclosure assembly 32 may be of any suitable construction, for example, plastic, metal, etc. Additionally and/or alternatively, housing 12 may include a window 34 in housing 12 and/or enclosure assembly 32 that may be configured to permit visual inspection of the solution, wherein cloudiness indicating product which should be discarded.

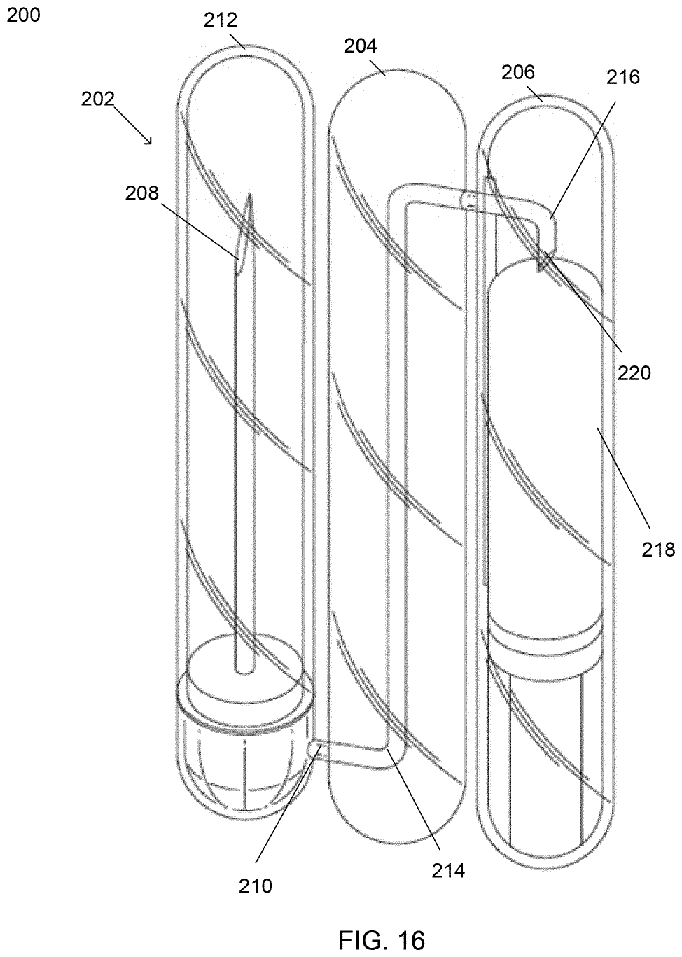

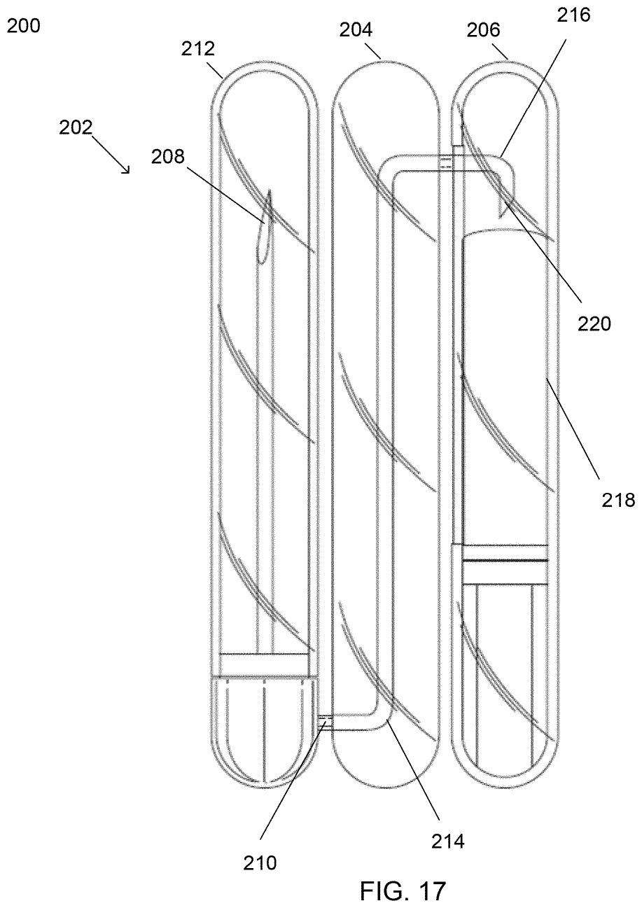

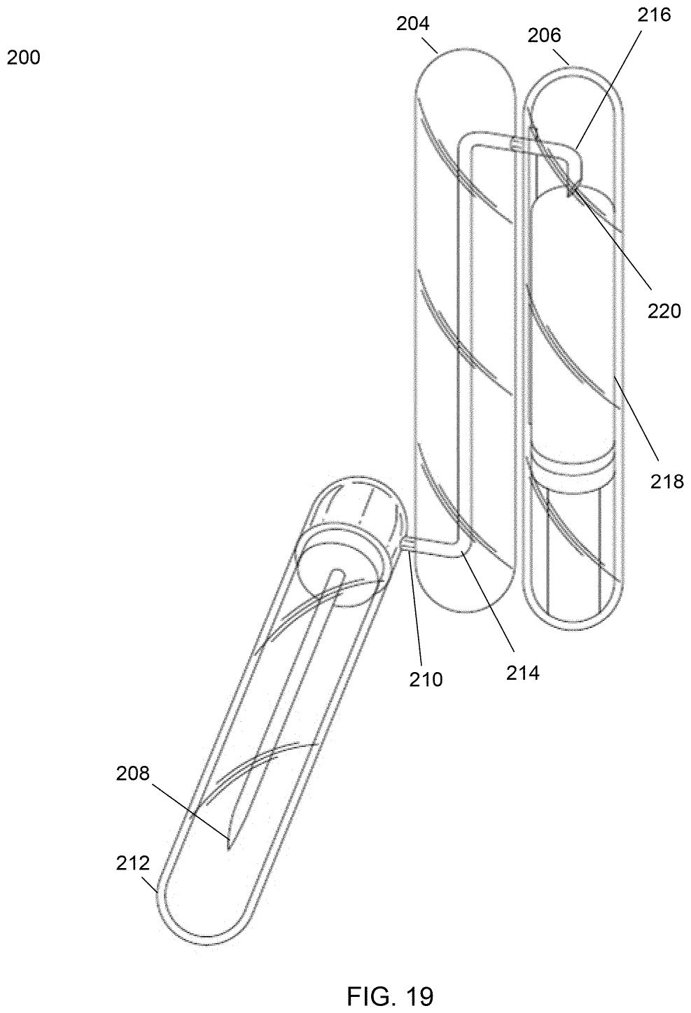

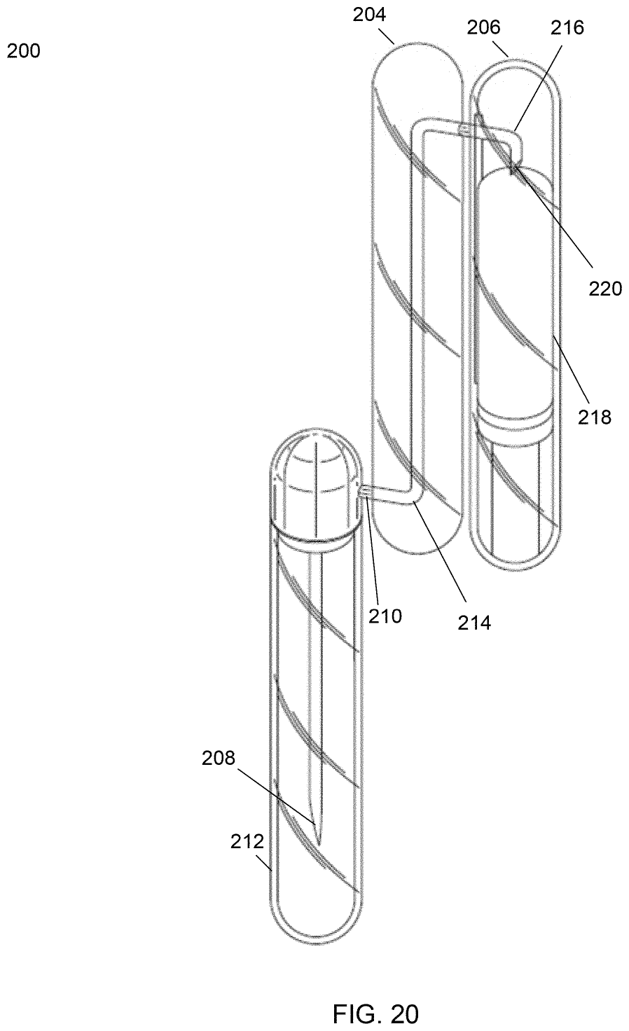

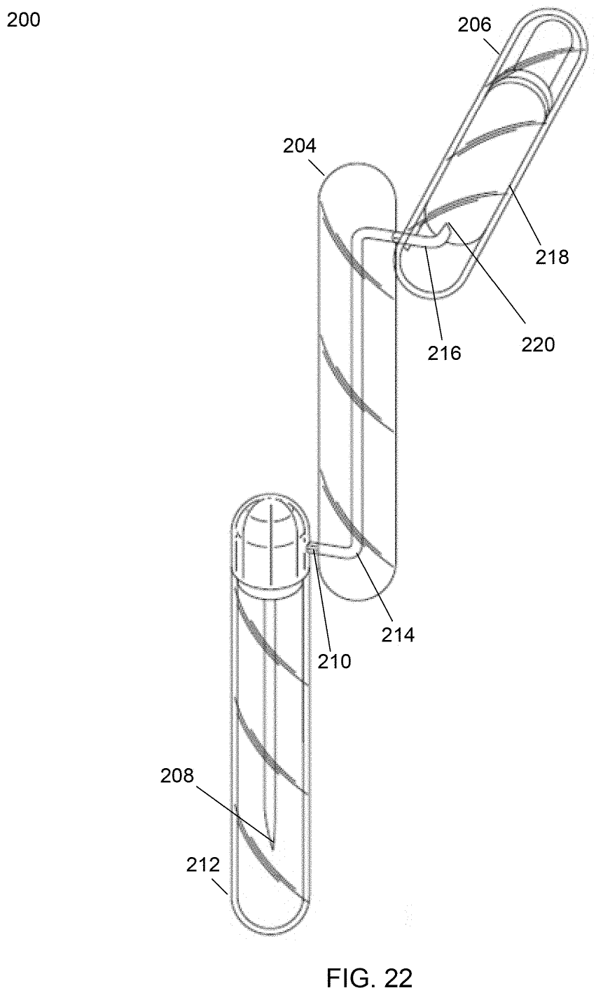

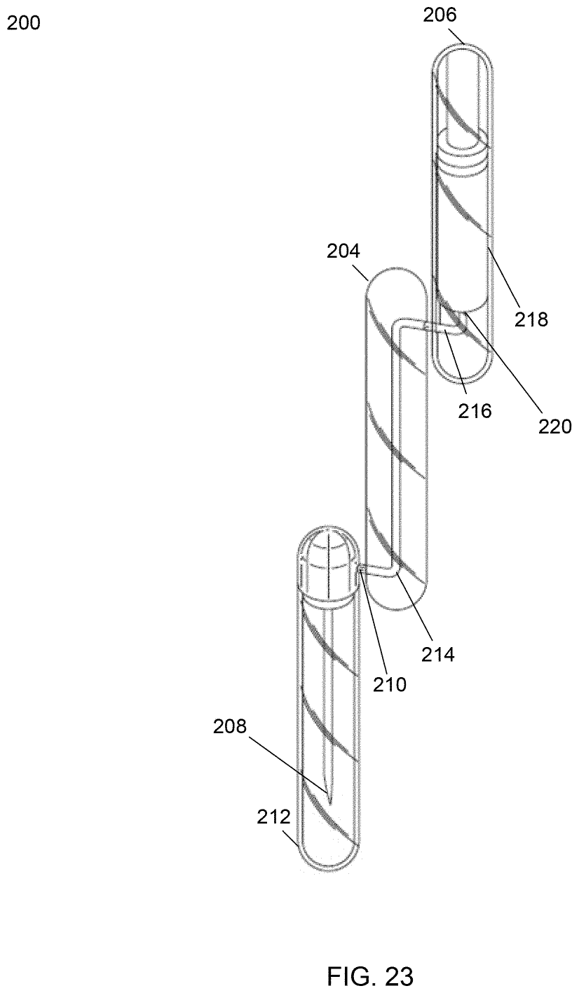

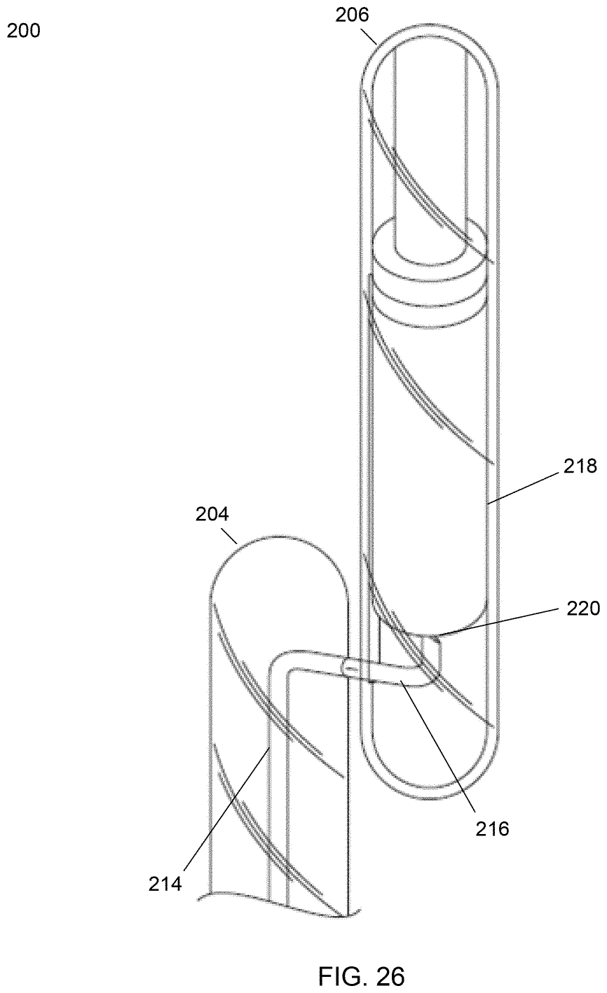

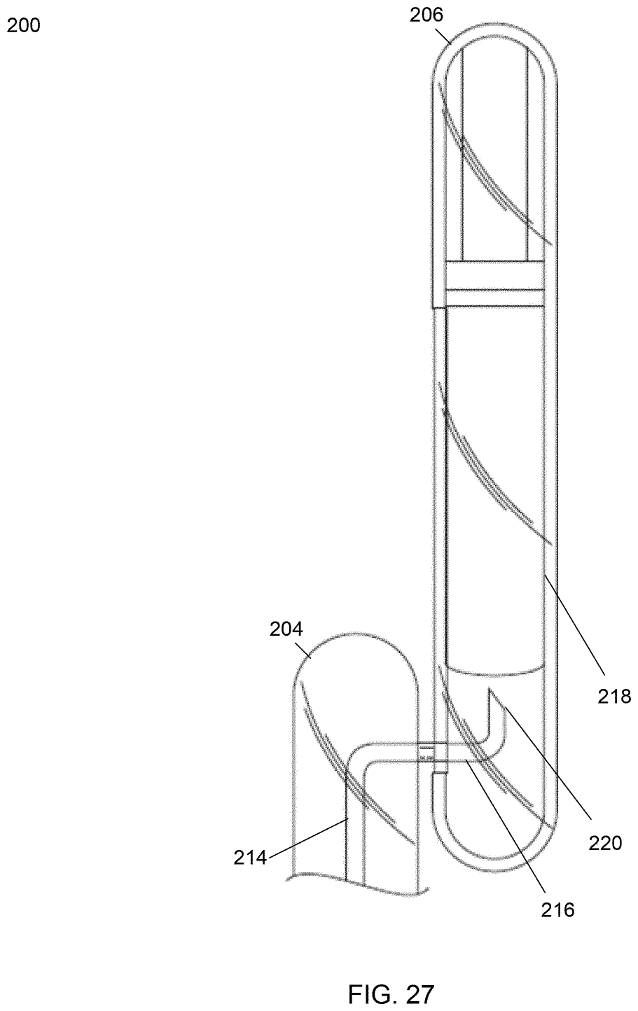

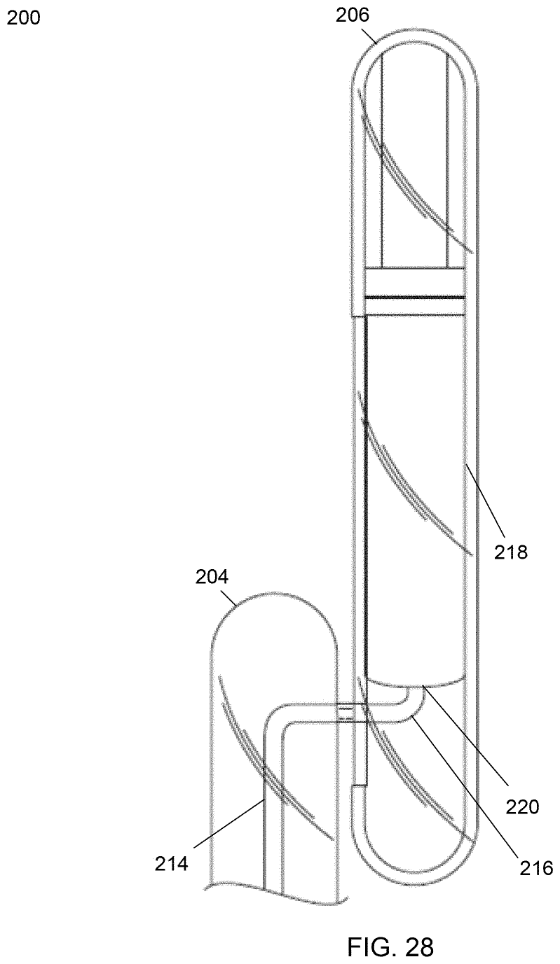

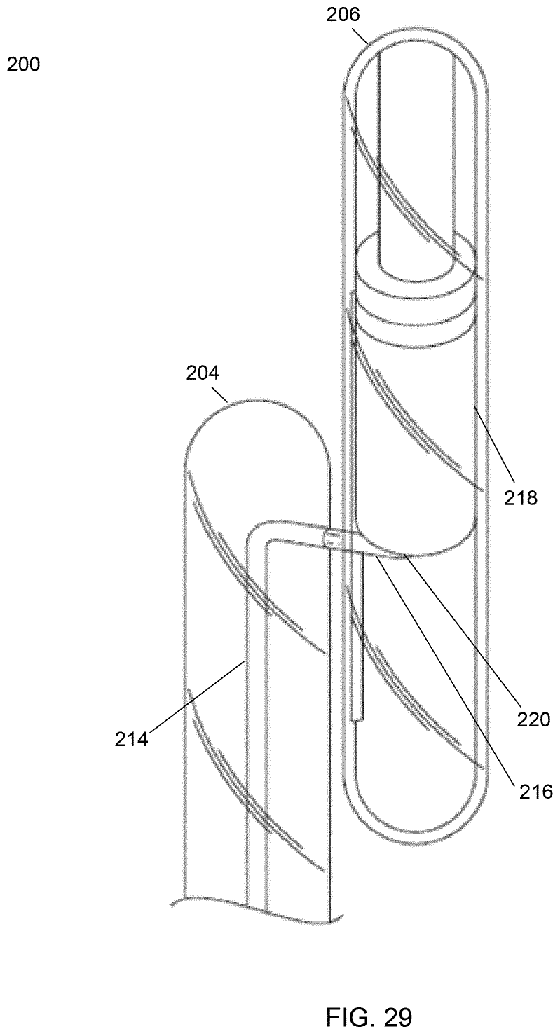

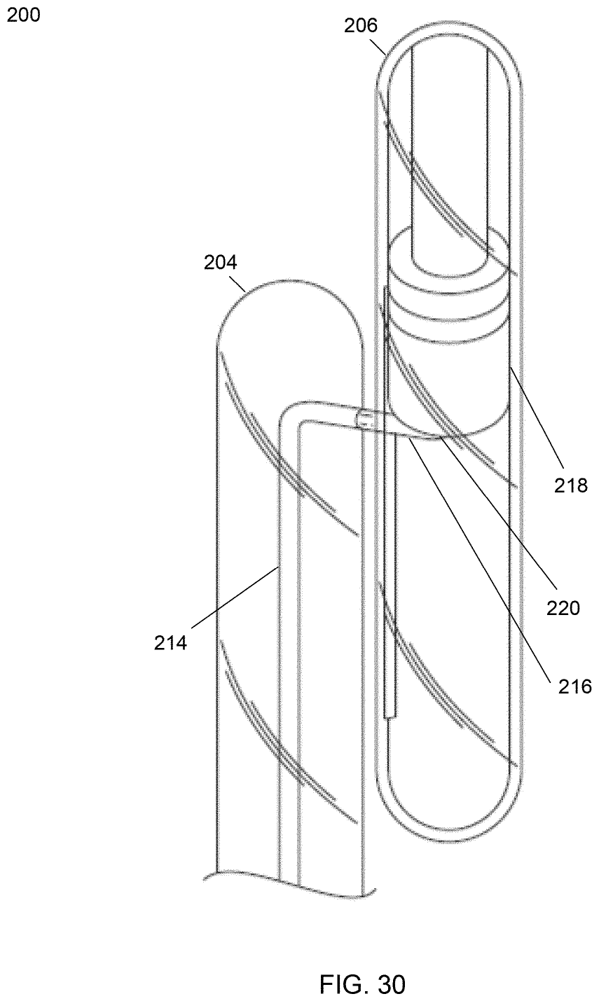

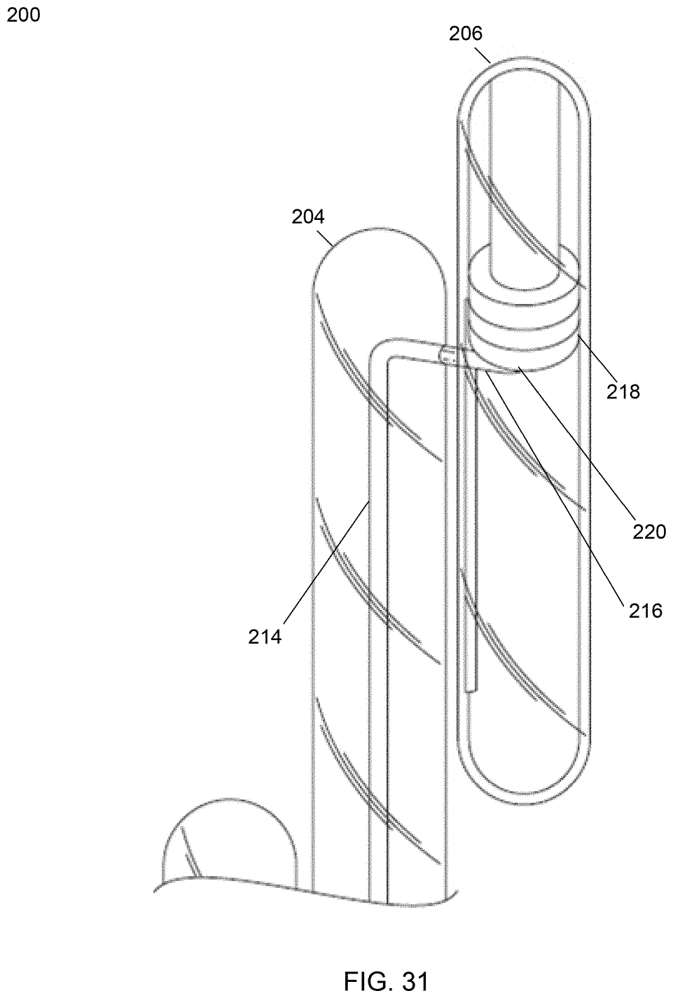

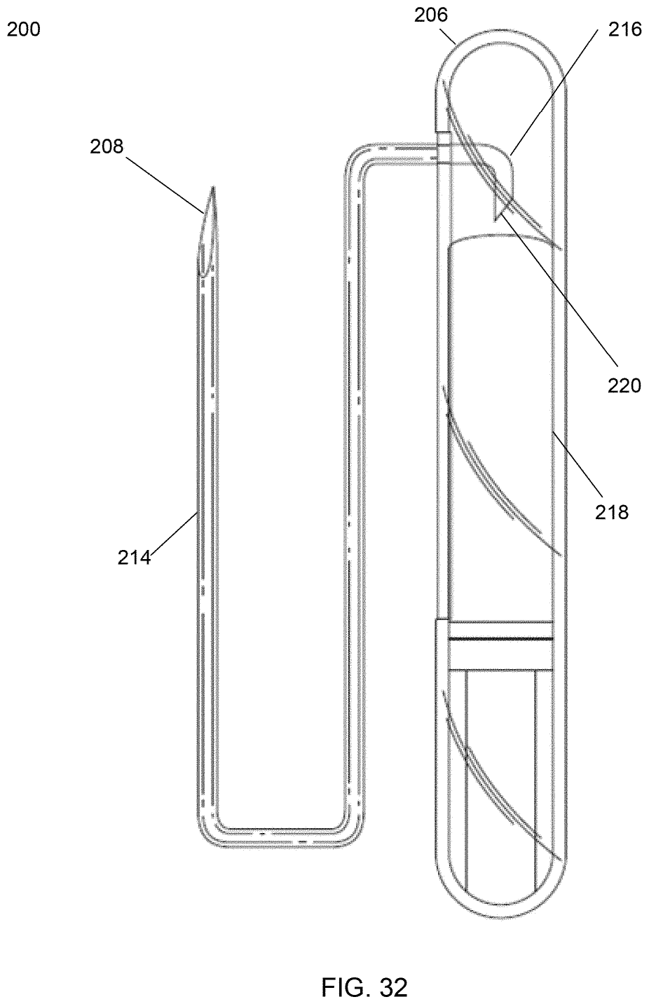

Referring now to FIGS. 16-32, additional embodiments consistent with the teachings of the present disclosure are provided. These figures depict a fluid dispensing apparatus 200, which includes first housing portion 202, second housing portion 204, and third housing portion 206. In some embodiments, first housing portion 202 may include injection needle 208 connected to a first fluid conduit 210 and also removable needle cap 212. Second housing portion 204 may include second fluid conduit 214. Third housing portion 206 may include third fluid conduit 216. Third housing portion 206 may further include vial 218, which may contain pre-dosed medication, and a vial piercing needle tip 220. In some embodiments, first housing portion 202 may be configured to rotate to connect to the second housing portion 204 and the second housing portion 204 may be configured to rotate to connect to third housing portion 206.

In some embodiments, second housing portion 204 and vial piercing tip 220 may be configured to operate together to puncture the vial 218, which may contain pre-dosed medication. In operation, fluid may be dispensed from third fluid conduit 216 through the second fluid conduit 214 and the first fluid conduit 210 before reaching injection needle 208. Third housing portion 206 may be configured to slide down the second housing portion 204 to dispense fluid.

In some embodiments, fluid conduits 210, 214, and 216 may be made out of any suitable material. Some materials may include, but are not limited to, sterile, metal materials. Injection needle 208 and vial piercing needle tip 220 may also be constructed out of any suitable material such as various metals, etc.

As shown in FIGS. 16-32, fluid dispensing apparatus 200 includes a foldable design, similar to a carpenter's folding ruler, that uses an internal sliding mechanism to actuate the internal plunger, minimizing size and maximizing portability. As shown in the figures, fluid dispensing apparatus 200 may include a plurality of separate housings connected by internal plumbing. The embodiments shown in the figures depict three separate housings but this is provided merely by way of example. The first housing portion 202 may function as a removable needle cap 212 and may be connected at one end via plumbing and a rotating mechanism to the center or second housing portion 204. The center housing portion 204 contains plumbing to connect to third housing portion 206 on the opposite side and opposite end via a rotating mechanism. Third housing portion 206 contains a vial piercing needle tip 220 that rotates into position. At time of use, the center housing portion 204 and piercing tip 220 may be configured to slide along a channel on the third housing portion 206, allowing for puncture of the medication vial 218 and plunging of vial to push adequate volume medication along the entire device plumbing to ultimately deliver the prescribed dose. Vial volume may consist of a volume to be administered plus residual plumbing volume.

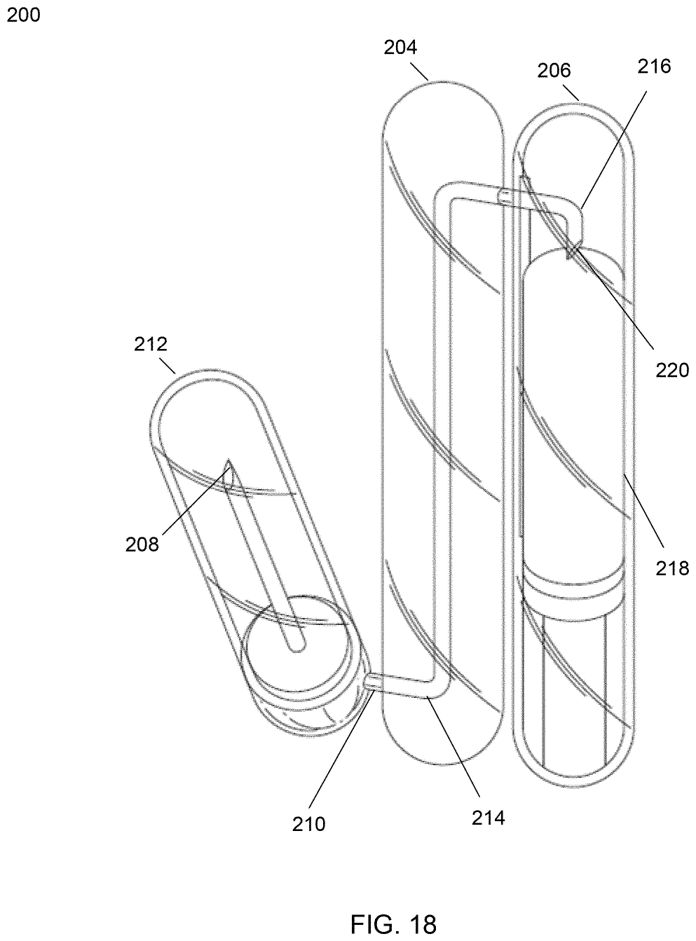

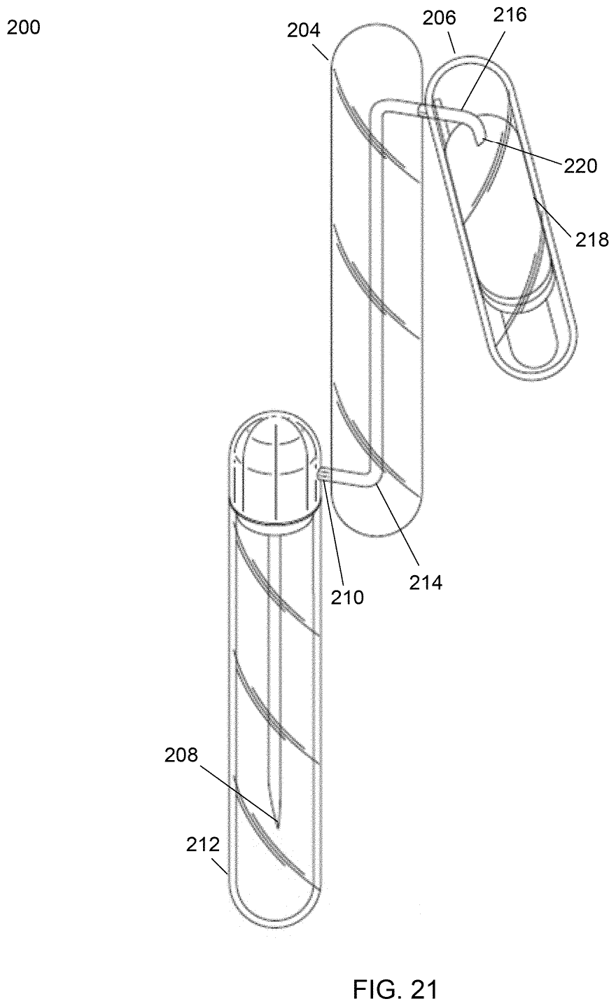

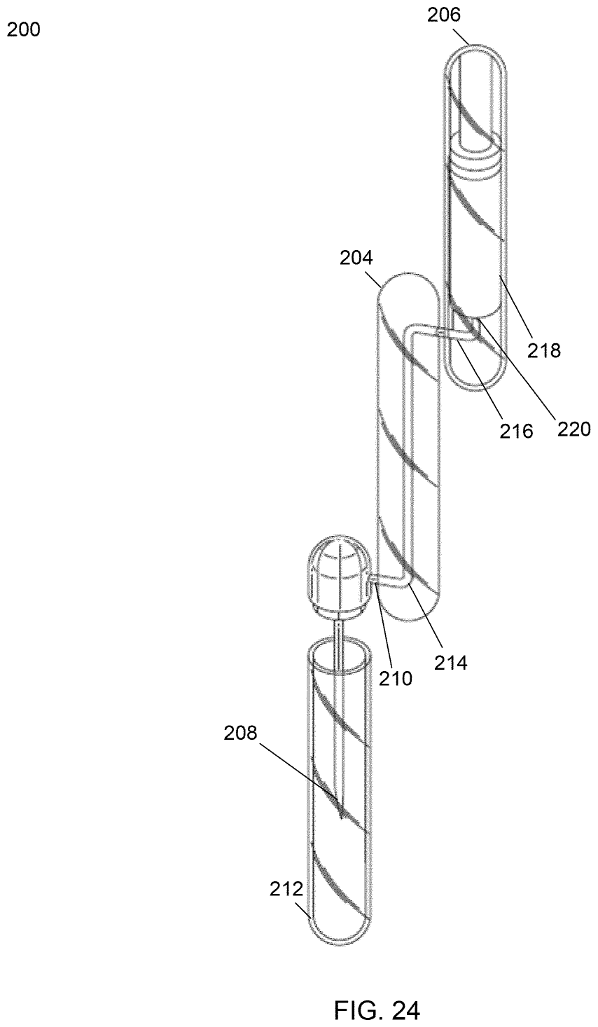

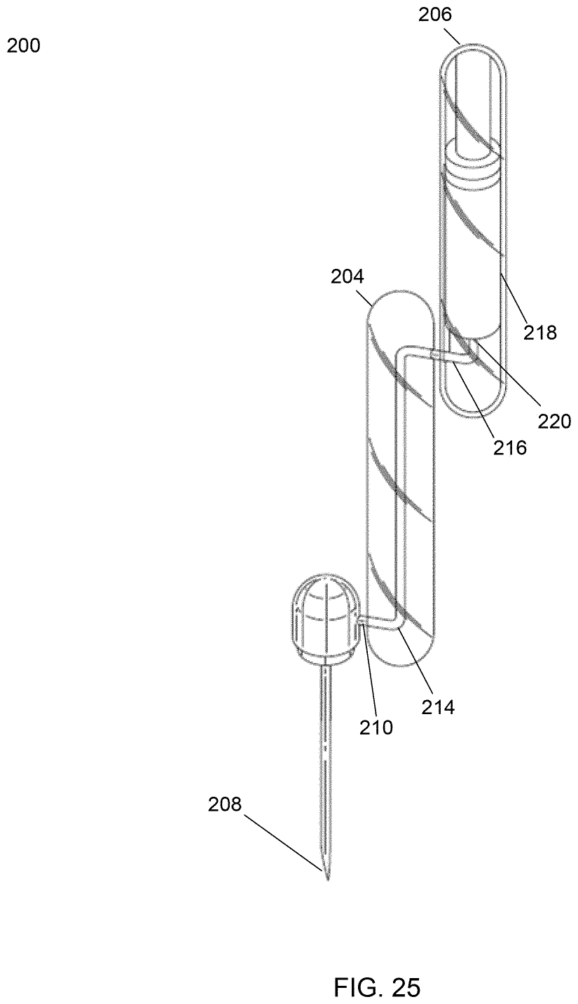

In operation, and referring again to FIGS. 18-20, first housing portion 202 may be rotated with respect to second housing portion 204. Similarly, third housing portion 206 may be rotated in a similar fashion as is shown in FIGS. 21-23. At this point, removable needle cap 212 may be removed as is shown in FIGS. 24-25. During injection second housing portion 204 may be configured to slide along third housing portion 206. Vial piercing tip 220 may pierce the vial membrane. Continued sliding then pushed vial down onto a stationary plunger, which depresses a movable stopper the back end of the vial, increasing the pressure, causing liquid to flow through the plumbing. Accordingly, the plunger may fit into the vial when activated. Alternatively, medication could be contained within a compressible sac instead of hard walled vial, device functioning as depicted.

In operation, for example in the event of a medical emergency, fluid dispensing apparatus 200 may be used by unfolding the housing portions 202, 204, and/or 206, removing needle cap 212, inserting into muscle or skin, then injecting the medication by sliding the third housing portion 206 down the center housing 204.

In some embodiments, the size of fluid dispensing apparatus 10, 200, etc. may be determined by the needle length required for intramuscular injection (e.g., 1 to 1.5 inches, etc.) as well as by the volume of medication. Any suitable dosing amount may be used without departing from the scope of the present disclosure. Standard dosing of epinephrine for example: 0.3 mg for adults or children>30 kg, and 0.15 mg for children<30 kg. The industry standard concentration is 1:1000 solution, therefore 0.3 mg=0.3 ml, and 0.15 mg=0.15 ml.

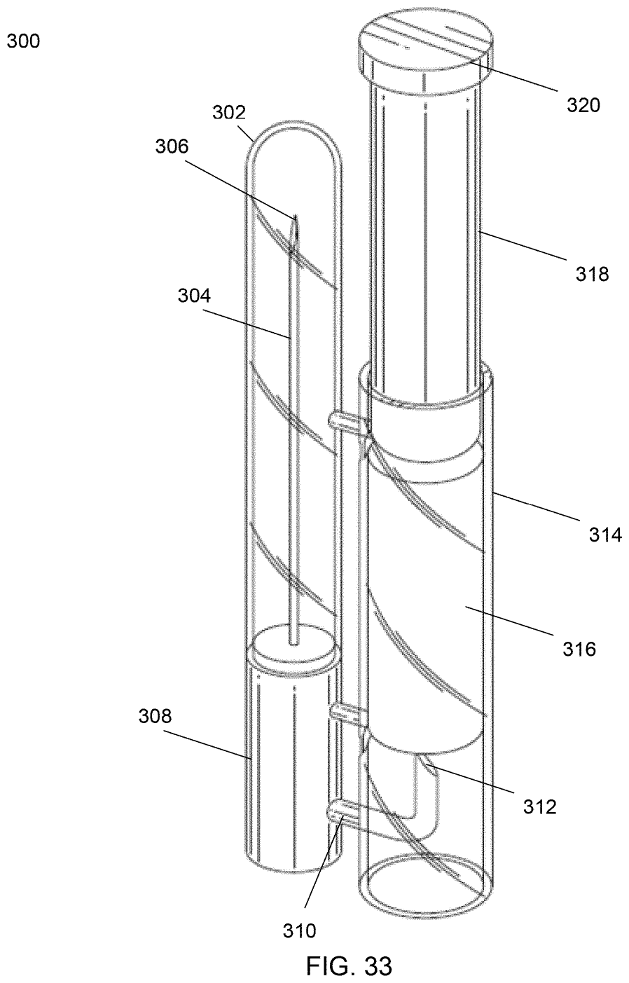

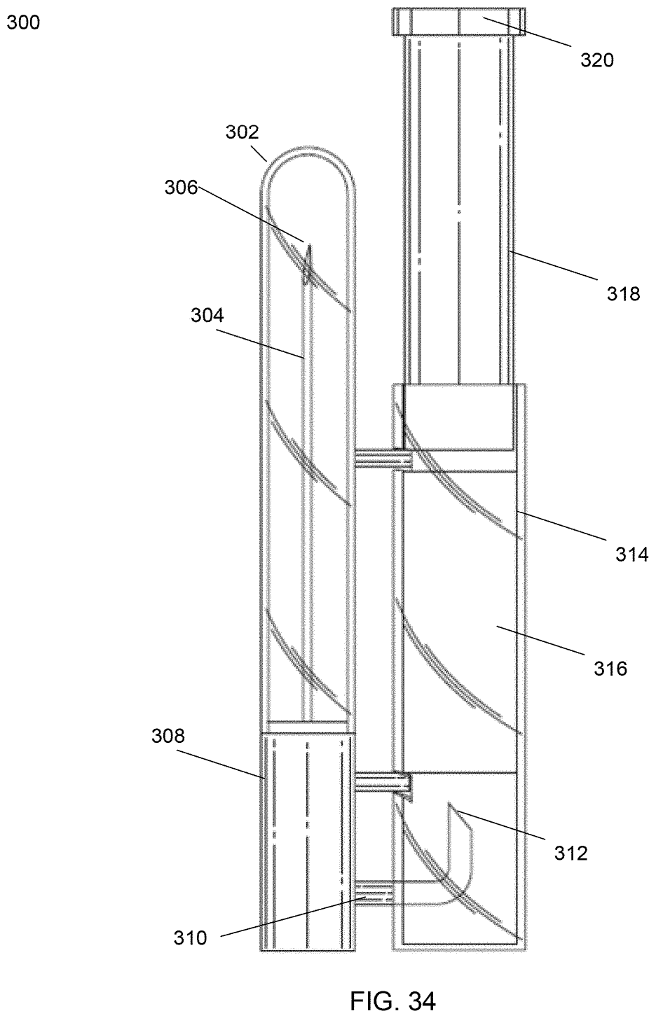

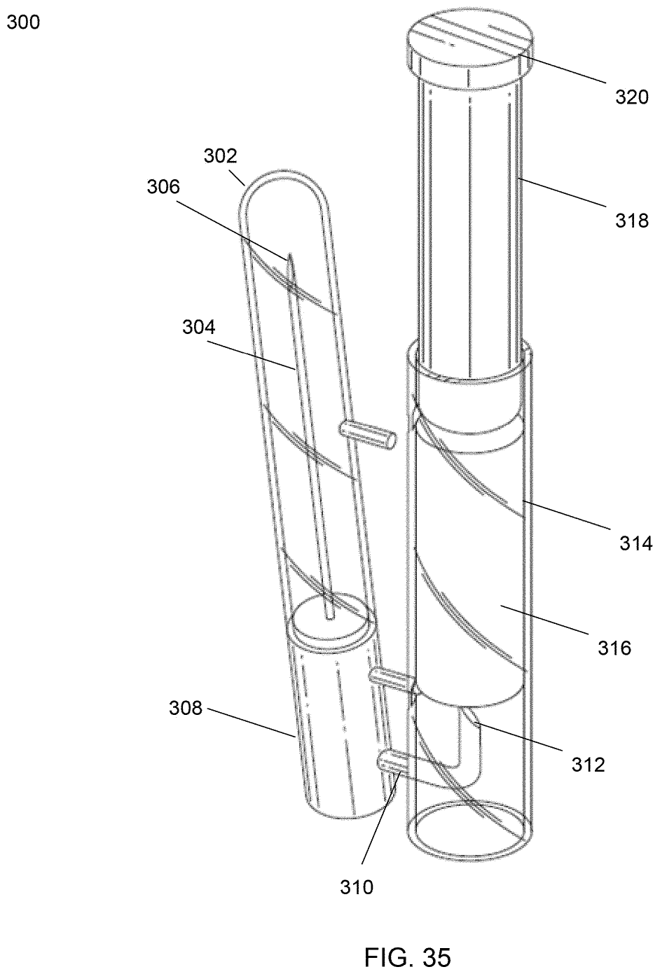

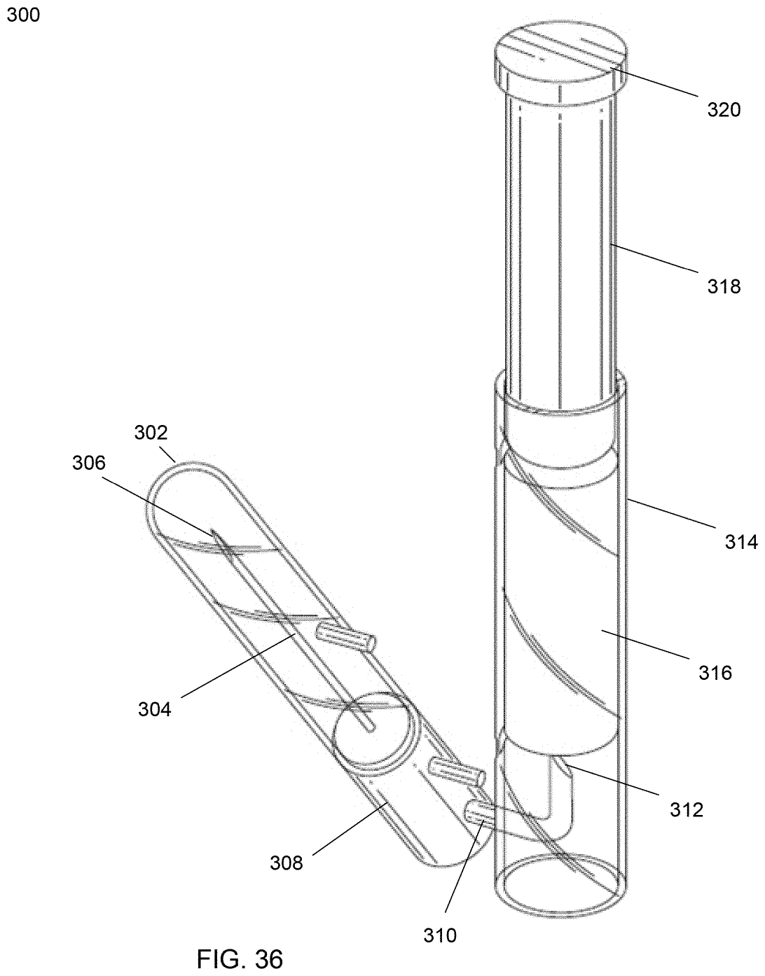

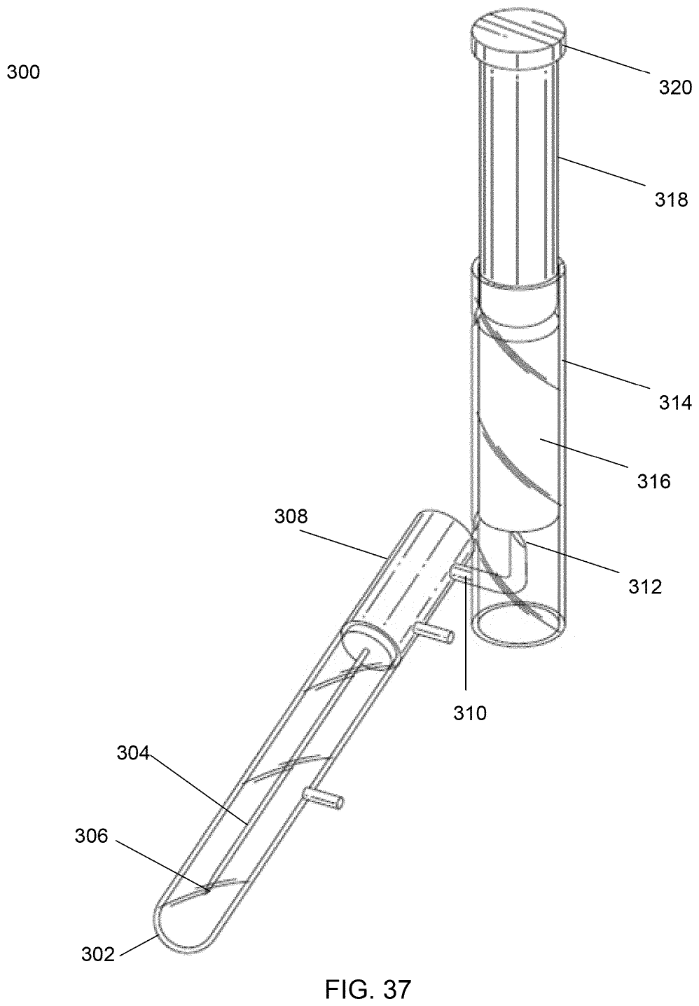

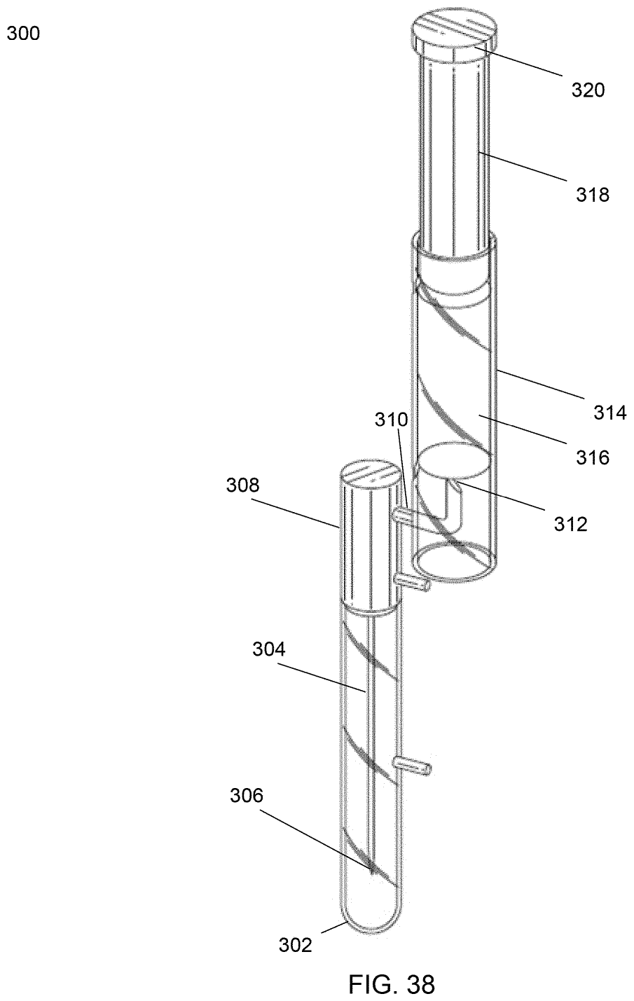

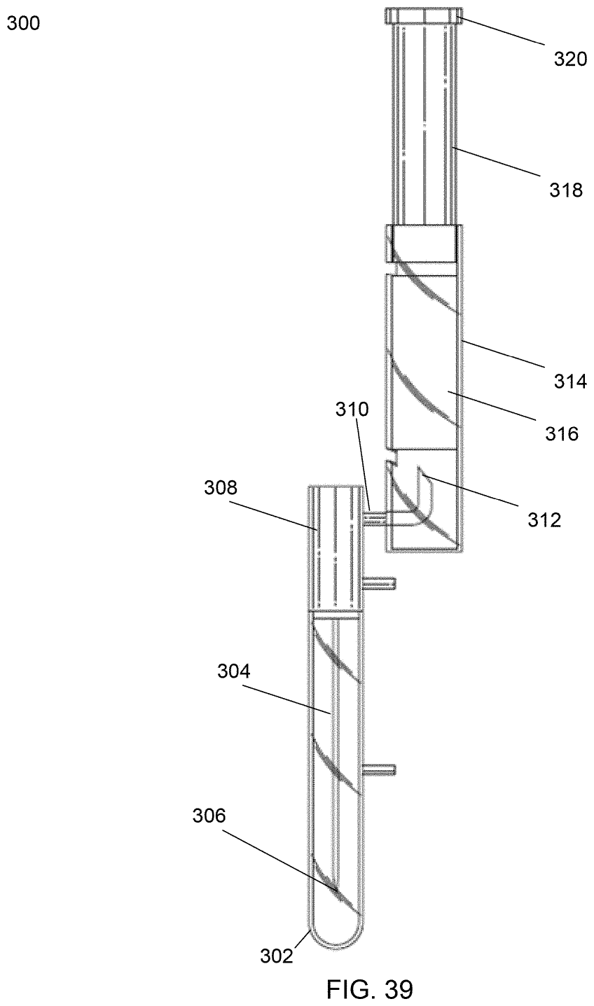

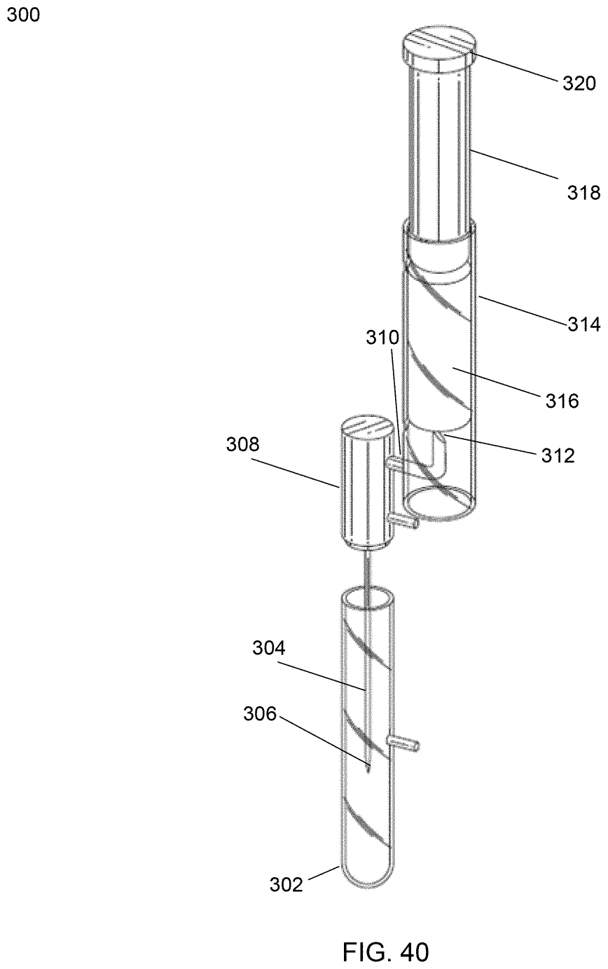

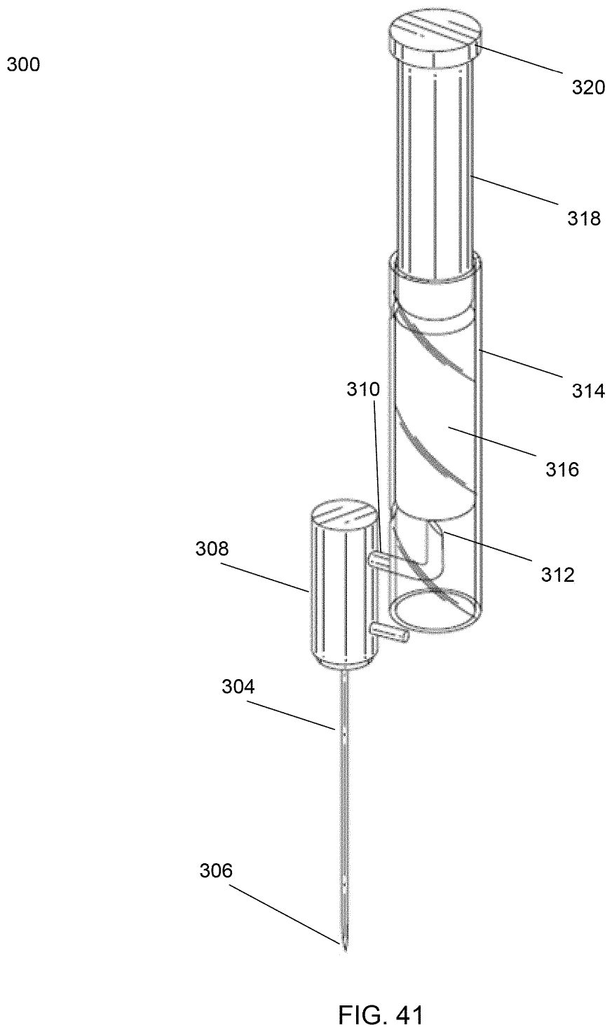

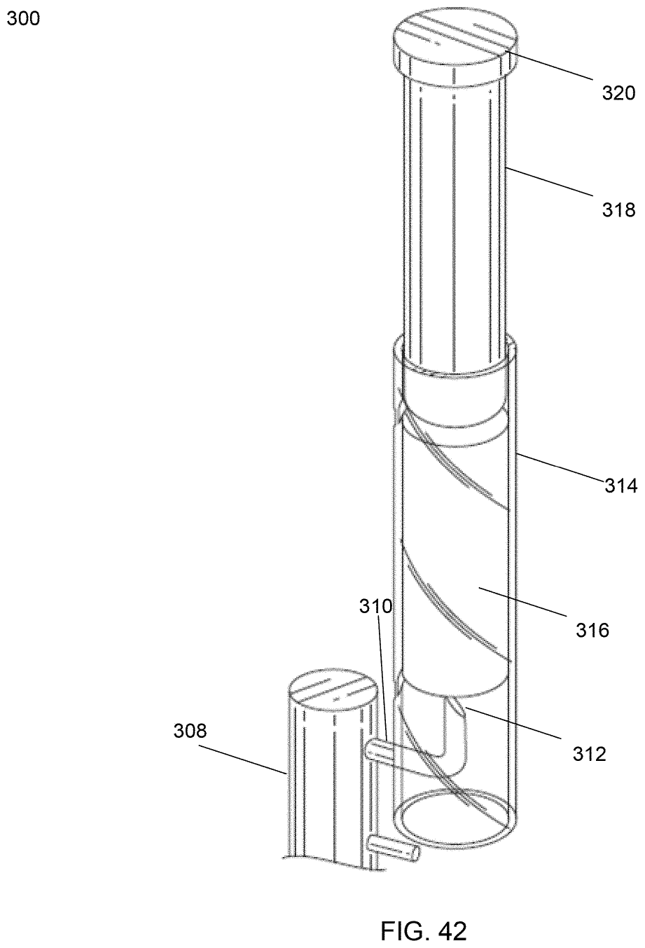

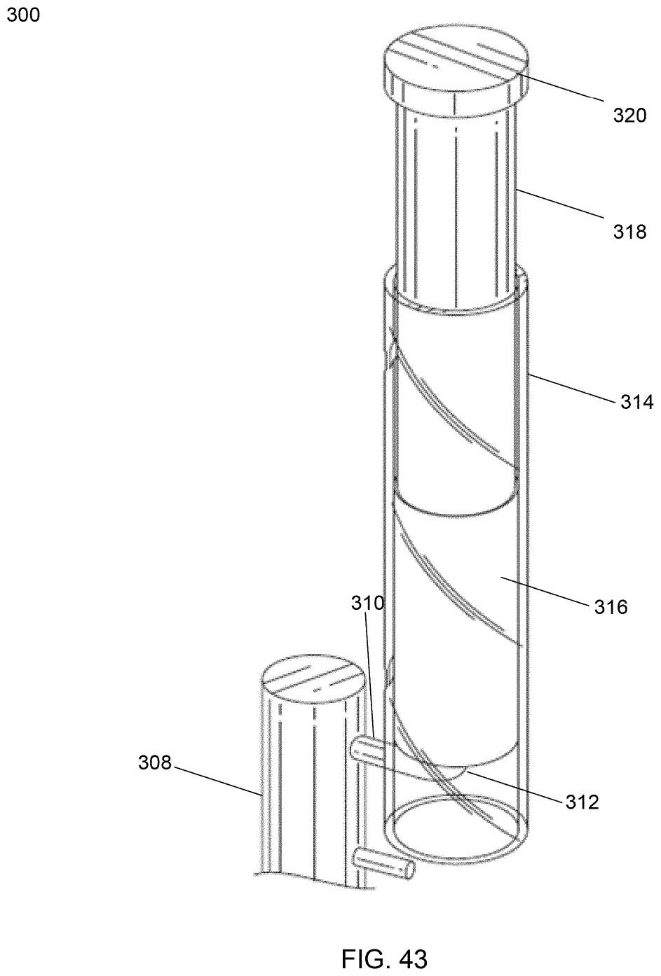

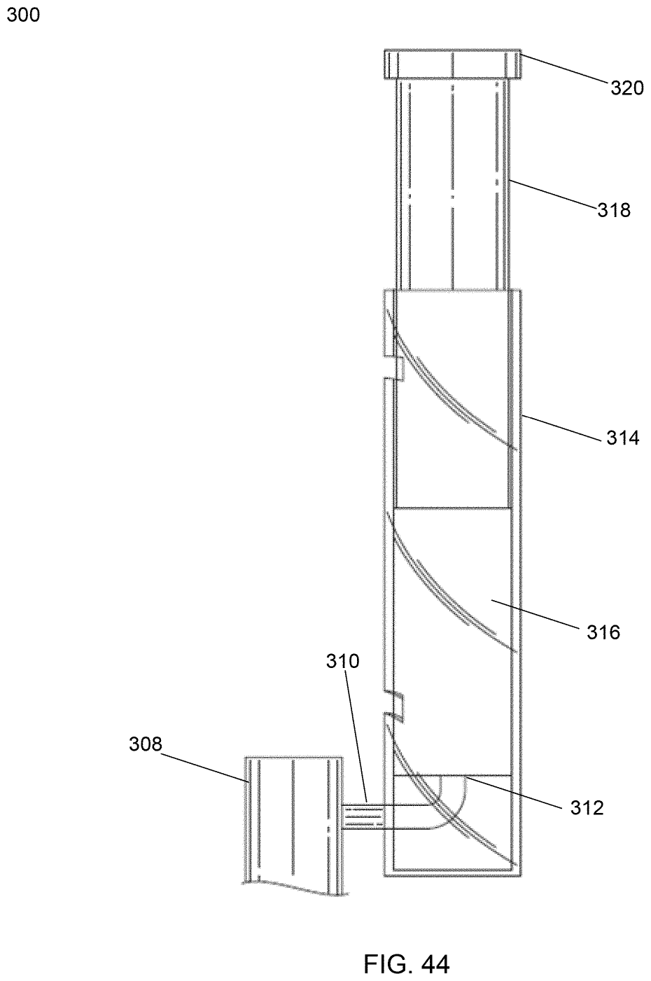

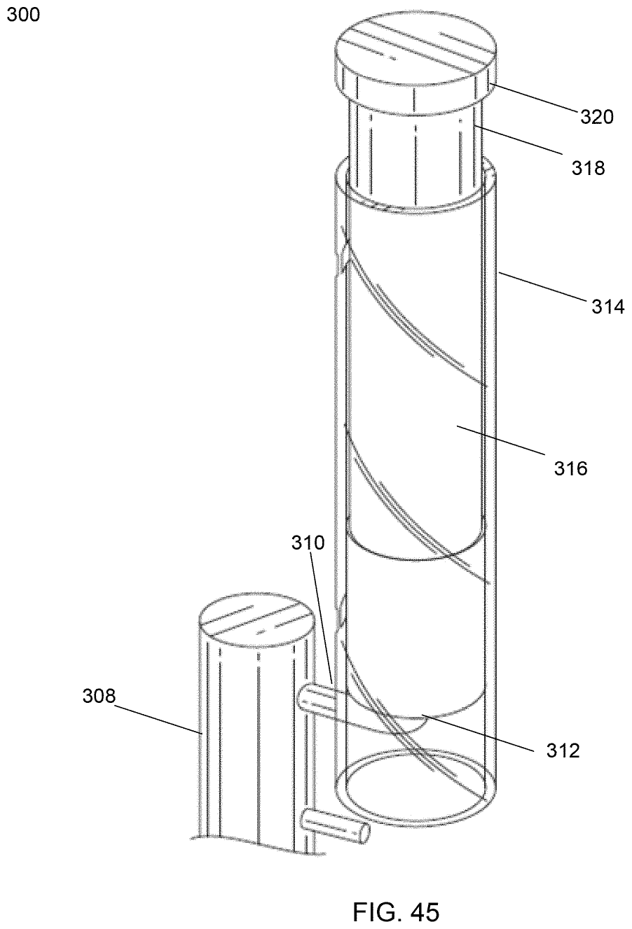

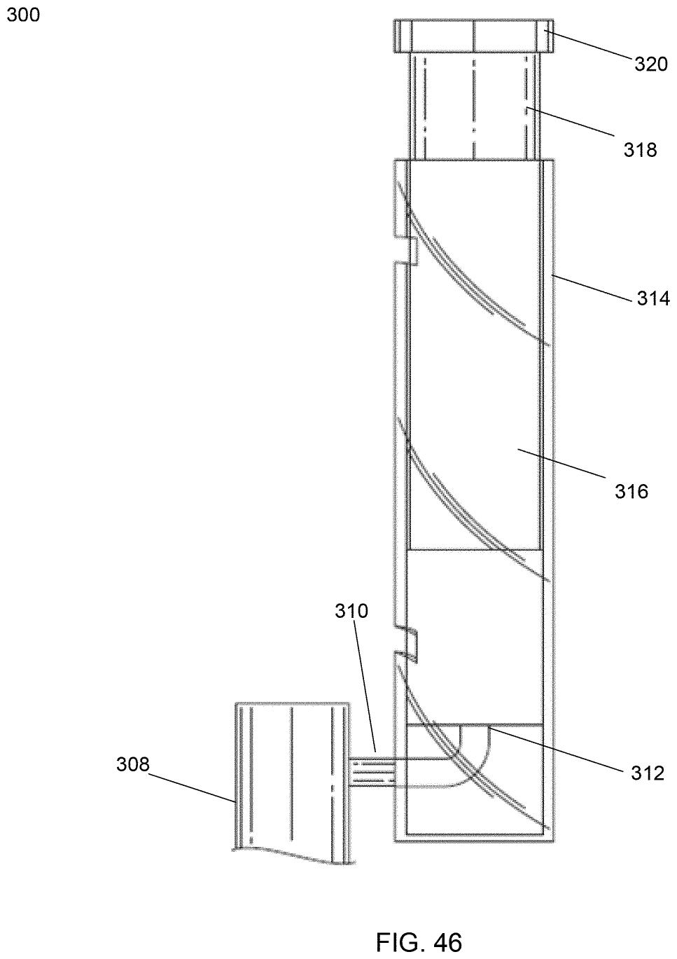

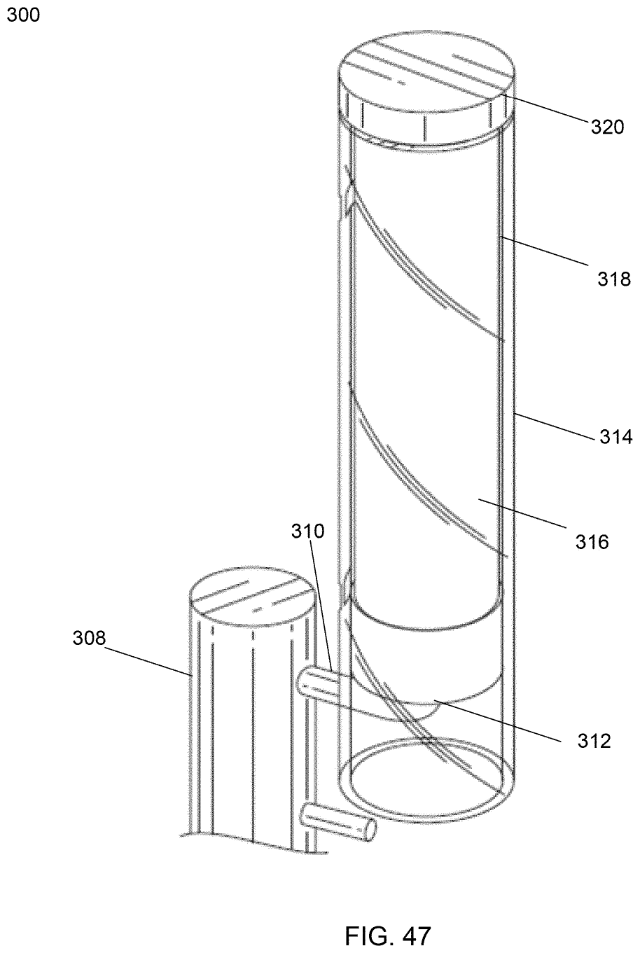

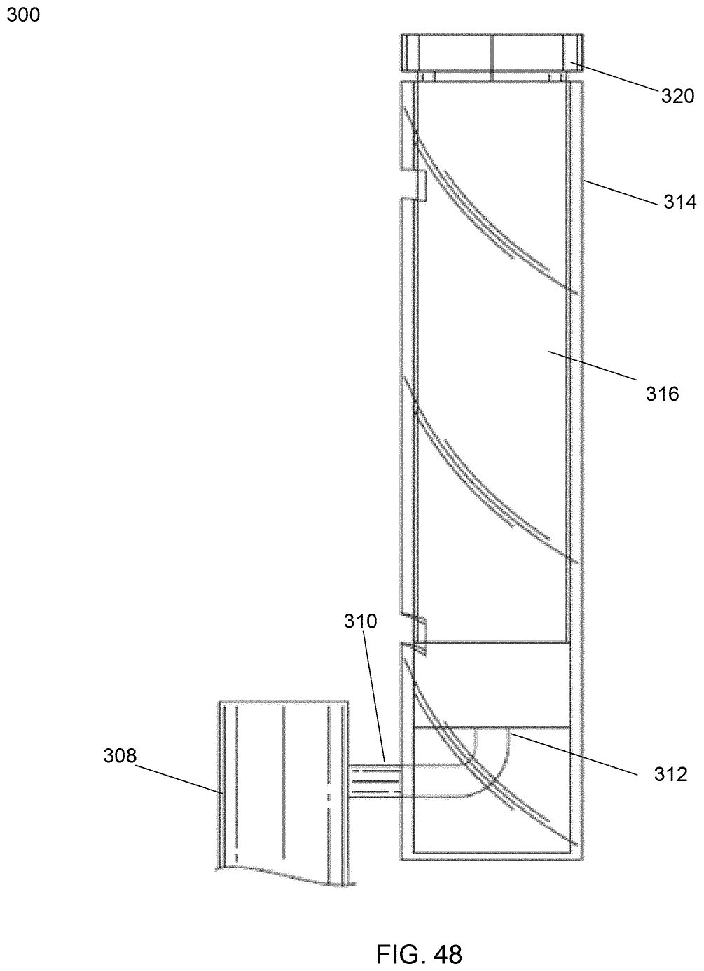

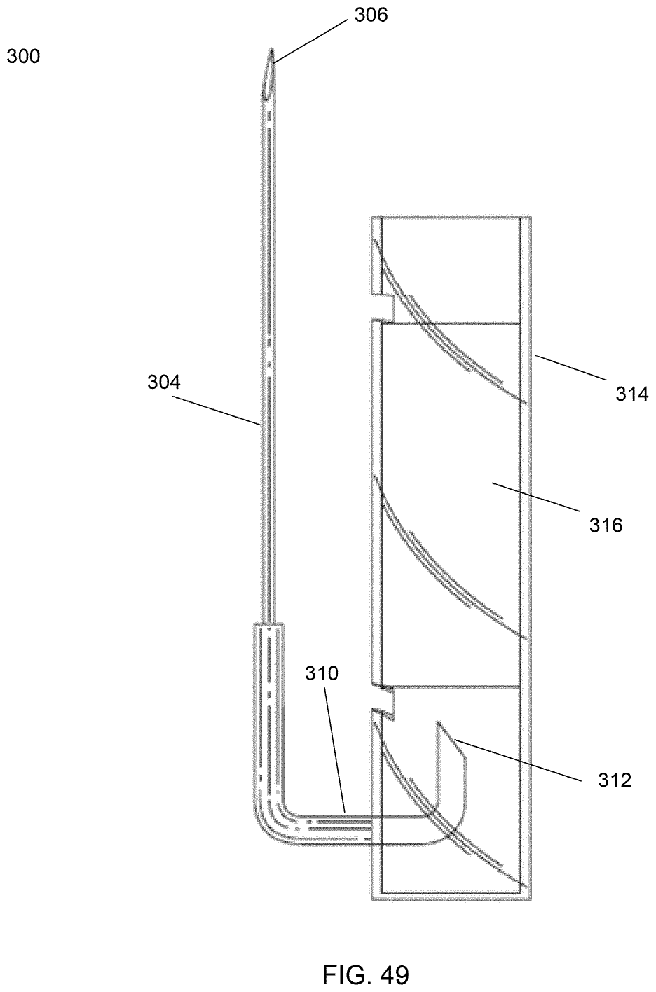

Referring now to FIGS. 33-49, additional embodiments consistent with the teachings of the present disclosure are provided. In some embodiments, fluid dispensing apparatus 300 may include a novel button shape and foldable needle design, that uses manual thumb pressure to cause piercing of internal medication vial and subsequent dispensation of medication after needle has been moved into place and needle guard removed, minimizing size and maximizing portability. In some embodiments, the needle guard may also functions as attachment point for necklace.

In some embodiments, fluid dispensing apparatus 300 may include removable needle cap 302, which may be configured to protect needle 304 having needle tip 306. Needle 304 may be operatively connected with a base portion 308. Base portion 308 may include a fluid conduit 310, which may allow for fluid transport through vial piercing end 312. Vial piercing end 312 may be contained within housing 314. Housing 314 may include drug storage compartment, which may be configured to house medication such as in the vials 316 discussed above. Housing 314 may be in mechanical communication with an actuator 318. In operation, a user may depress button 320, which in turn forces actuator 318 to move in an axially direction along housing 314 and allow for the puncture of vial 316 by vial piercing end 312.

In some embodiments, prior to use, locking pins 322 and 324, attached to needle cap 302, may be configured to prevent accidental vial puncture and plunging. In operation, needle cap 302, needle tip 306 and base portion 308 may rotate 180 degrees on the axis of fluid conduit 310. Such movement may cause the locking pins 322 and 324 to disengage from housing 314. Needle cap 302 may then be removed and patient inserted needle 306 into appropriate muscle and begins applying manual thumb pressure to button 320. With the above disengagement of locking pin 324, the vial 316 may now be free to move within the housing 31. The plunger 318 may now be free to depress the vial down onto 312, and then continue plunging the stopper at the back end of the vial, causing liquid medication to flow along the plumbing 310, the needle 304 and into the patient's body.

In some embodiments the amount of available medication could be increased (e.g. doubled) by duplicating 310-320 on the other side of 308, and adding additional locking pins to 302 and 308.

In some embodiments, a fluid dispensing apparatus may include a small, double barreled device containing either 1 or 2 vials of 0.15 ml epinephrine (1 for pediatric, 2 for adult), with a central needle mounted at its base by a rotating plumbing system. The plumbing ends in two vial piercing tips that puncture the vials at time of actuation.

In some embodiments, the fluid dispensing apparatuses described herein would be small, easy to operate, and always on person, like jewelry (e.g. necklace or bracelet). It should be noted that the elimination of any spring mechanism from auto-injectors would permit optimization of size and portability. Proposed device would now be manual, but more likely to be carried. The elimination of any spring-loaded mechanism in favor of foldable mechanisms permit small size and portability to the point of wearing the device as jewelry, increasing likelihood of availability at time of emergency.

1. A fluid dispensing apparatus, comprising:
a housing including a vial containing pre-dosed medication;
an injection needle contained within a removable needle cap, the injection needle having a drug contact end and a patient contact end, the injection needle configured to puncture at both ends;
a hinge operatively connecting the housing and the injection needle, wherein the hinge provides movement from a first position wherein the drug contact end is not in contact with the pre-dosed medication to a second position where the drug contact end is in contact with the pre-dosed medication; and
an opening associated with a proximal end of the housing configured to receive the removable needle cap.

2. The fluid dispensing apparatus of claim 1, further comprising:
a sterilization cap configured to cover the housing and the injection needle.

3. The fluid dispensing apparatus of claim 1, further comprising:
a movable cover configured to restrict or allow access to an inner portion of the housing.

4. The fluid dispensing apparatus of claim 1, wherein the pre-dosed medication is selected from the group consisting of epinephrine.

5. The fluid dispensing apparatus of claim 1, wherein the housing is transparent.

6. The fluid dispensing apparatus of claim 1, wherein the drug contact end and the patient contact end of the needle are beveled.

7. The fluid dispensing apparatus of claim 1, further comprising:
at least one connector configured to allow a patient to wear the apparatus.

8. The fluid dispensing apparatus of claim 1, wherein the housing is at least one of rectangular and cylindrical.

9. A fluid dispensing method, comprising:
providing a housing including a vial containing pre-dosed medication;
connecting an injection needle contained within a removable needle cap to the housing using a hinge, the injection needle having a drug contact end and a patient contact end;
moving the injection needle from a first position wherein the drug contact end is not in contact with the pre-dosed medication to a second position where the drug contact end is in contact with the pre-dosed medication; and
receiving the removable needle cap at an opening associated with a proximal end of the housing.

10. The fluid dispensing method of claim 1, further comprising:
covering the housing and the injection needle with a sterilization cap.

11. The fluid dispensing method of claim 1, further comprising:
using a movable cover configured to restrict or allow access to an inner portion of the housing.

12. The fluid dispensing method of claim 1, wherein the pre-dosed medication is selected from the group consisting of epinephrine.

13. The fluid dispensing method of claim 1, wherein the housing is transparent.

14. The fluid dispensing method of claim 1, wherein the drug contact end and the patient contact end of the needle are beveled.

15. The fluid dispensing method of claim 1, further comprising:
connecting the apparatus with a patient using at least one connector.

16. The fluid dispensing method of claim 1, wherein the housing is at least one of rectangular and cylindrical.

17. A fluid dispensing apparatus, comprising:
a first housing portion including an injection needle connected to a first fluid conduit and a removable needle cap;
a second housing portion including a second fluid conduit; and
a third housing portion including a third fluid conduit, a vial containing pre-dosed medication, and a vial piercing needle tip, wherein the first housing portion is configured to rotate to connect to the second housing portion and the second housing portion is configured to rotate to connect to the third housing portion.

18. The fluid dispensing apparatus of claim 17 wherein the second housing and vial piercing tip operate together to puncture the vial containing pre-dosed medication.

19. The fluid dispensing apparatus of claim 17 wherein the third housing slides down the second housing to dispense fluid.

20. The fluid dispensing apparatus of claim 19 wherein fluid is dispensed from the third fluid conduit through the second fluid conduit and the first fluid conduit before reaching the injection needle.
